(19) United States
(12) Patent Application Publication
(10) Pub. No.: US 2003/0082103 A1
(43) Pub. Date: May 1, 2003

(54) Targeted therapeutic lipid constructs having cell surface targets

(75) Inventors: Charles Aaron Wartchow, San Carlos, CA (US); John S. Pease, Los Altos, CA (US); Zhi Min Shen, Palo Alto, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: TARGESOME, INC.

(21) Appl. No.: 10/262,576

(22) Filed: Oct. 1, 2002

Related U.S. Application Data
(63) Continuation-in-part of application No. 09/976,254, filed on Oct. 11, 2001.
(60) Provisional application No. 60/239,684, filed on Oct. 11, 2000.
(60) Provisional application No. 60/326,310, filed on Oct. 1, 2001.

(51) Int. Cl. A61K 51/00; C07F 5/00

(52) U.S. Cl. 424/1.53; 534/11

(57) ABSTRACT

Novel therapeutic lipid constructs comprising a polymerized liposome, an anti-cell surface targeting agent, and a radiotherapeutic metal ion are disclosed.

### CROSS-REFERENCE TO RELATED APPLICATIONS

This application is a continuation-in-part of U.S. patent application Ser. No. 09/976,254 entitled “Targeted Therapeutic Agents,” filed Oct. 11, 2001, which claims the benefit of U.S. Provisional Patent Application No. 60/239,684 entitled “Vascular-Targeted Therapeutic Agents” filed Oct. 11, 2000. This application also claims the benefit of U.S. Provisional Patent Application No. 60/326,310 entitled “Targeted Therapeutic Lipid Constructs Having Cell Surface Targets” filed Oct. 1, 2001.

### FIELD OF THE INVENTION

This invention relates to therapeutic and imaging agents which are comprised of a targeting entity, a linking carrier, and optionally, a therapeutic or treatment entity. The preferred agents of the present invention comprise a lipid construct, vesicle, liposome, or polymerized liposome. The therapeutic or treatment entity may be associated with the agent by covalent or non-covalent means. In some cases, the therapeutic or treatment entity is a prodrug, chemotherapeutic, toxin, radioisotope, or and a gene encoding a protein that exhibits cell toxicity. Preferably, the agent is further comprised of a stabilizing entity which imparts additional advantages to the therapeutic or imaging agent. The stabilizing entity may be associated with the agent by covalent or non-covalent means. Preferably, the stabilizing entity is dextran, which preferably forms a coating on the surface of the lipid construct, vesicle, liposome, or polymerized liposome. In preferred embodiments the linking carrier is a polymerized liposome. The linking carrier imparts additional advantages to the therapeutic agents, which are not provided by conventional linking methods.

### BACKGROUND OF THE INVENTION

Cancer remains one of the leading causes of death in the industrialized world. In the United States, cancer is the second most common cause of death after heart disease, accounting for approximately one-quarter of the deaths in 1997. Clearly, new and effective treatments for cancer will provide significant health benefits. Among the wide variety of treatments proposed for cancer, targeted therapeutic agents hold considerable promise. In principle, a patient could tolerate much higher doses of a cytotoxic agent if the cytotoxic agent is targeted specifically to cancerous tissue, as healthy tissue should be unaffected or affected to a much smaller extent than the pathological tissue.

Due to the high specificity of monoclonal antibodies, antibodies coupled to cytotoxic agents have been proposed for targeted cancer treatment therapies. Solid tumors, in particular, express certain antigens, on both the transformed cells comprising the tumor and the vasculature supplying the tumors, which are either unique to the tumor cells and vasculature, or overexpressed in tumor cells and vasculature in comparison to normal cells and vasculature. Thus, linking an antibody specific for a tumor antigen, or a tumor vasculature antigen, to a cytotoxic agent, should provide high specificity to the site of pathology. One group of such antigens is a family of proteins called cell adhesion molecules (CAMS), expressed by endothelial cells during a variety of physiological and disease processes. Reisfeld, “Monoclonal Antibodies in Cancer Immunotherapy,” Laboratory Immunology II, (1992) 12(2):201-216, and Archelos et al., “Inhibition of Experimental Autoimmune Encephalomyelitis by the Antibody to the Intercellular Adhesion Molecule ICAM-1,” Ann. of Neurology (1993) 34(2):145-154. Multiple endothelial ligands and receptors, including CAMs, are known to be upregulated during various pathologies, such as inflammation and neoplasia, and hence are attractive candidates for targeting strategies.

Integrins as Targets

Other potential targets are integrins. Integrins are a group of cell surface glycoproteins that mediate cell adhesion and therefore are mediators of cell adhesion interactions that occur in various biological processes. Integrins are heterodimers composed of noncovalently linked α and β polypeptide subunits. Currently at least eleven different a subunits have been identified and at least six different β subunits have been identified. The various a subunits can combine with various β subunits to form distinct integrins. The integrin identified as αᵥβ₃ (also known as the vitronectin receptor) has been identified as an integrin that plays a role in various conditions or disease states including but not limited to tumor metastasis, solid tumor growth (neoplasia), osteoporosis, Paget's disease, humoral hypercalcemia of malignancy, angiogenesis, including tumor angiogenesis, anti-angiogenesis, retinopathy,macular degeneration, arthritis, including rheumatoid arthritis, periodontal disease, psoriasis and smooth muscle cell migration (e.g., restenosis). Additionally, it has been found that such integrin inhibiting agents would be useful as antivirals, antifungals and antimicrobials. Thus, therapeutic agents that selectively inhibit or antagonize αᵥβ₃ would be beneficial for treating such conditions. It has been shown that the αᵥβ₃ integrin binds to a number of Arg-Gly-Asp (RGD) containing matrix macromolecules, such as fibrinogen (Bennett et al., Proc. Natl. Acad. Sci. USA, Vol. 80 (1983) 2417), fibronectin (Ginsberg et al., J. Clin. Invest., Vol. 71 (1983) 619-624), and von Willebrand factor (Ruggeri et al., Proc. Natl. Acad. Sci. USA, Vol. 79 (1982) 6038). Compounds containing the RGD sequence mimic extracellular matrix ligands so as to bind to cell surface receptors. However, it is also known that RGD peptides in general are non-selective for RGD dependent integrins. For example, most RGD peptides that bind to αᵥβ₃ also bind to αᵥβ₅, αᵥβ₁, and α_{IIb}β_{IIIa}. Antagonism of platelet α_{IIb}β_{IIIa} (also known as the fibrinogen receptor) is known to block platelet aggregation in humans.

A number of anti-integrin antibodies are known. Doerr, et al., J. Biol. Chem. 1996 271:2443 reported that a blocking antibody to αᵥβ₅ integrin in vitro inhibits the migration of MCF-7 human breast cancer cells in response to stimulation from IGF-1. Gui et al., British J. Surgery 1995 82:1192, report that antibodies against αᵥβ₁ and αᵥβ₅ inhibit in vitro chemoinvasion by human breast cancer carcinoma cell lines Hs578T and MDA-MB-231. Lehman et al., Cancer Research 1994 54:2102 show that a monoclonal antibody (69-6-5) reacts with several αᵥ integrins including αᵥβ₃ and inhibited colon carcinoma cell adhesion to a number of substrates, including vitronectin. Brooks et al., Science 1994 264:569 show that blockade of integrin activity with an anti-αᵥβ₃ monoclonal antibody inhibits tumor-induced angiogenesis of chick chorioallantoic membranes by human M21-L melanoma fragments. Chuntharapai, et al., Exp. Cell. Res. 1993 205:345 discloses monoclonal antibodies 9G2.1.3 and IOC4.1:3 which recognize the αᵥβ₃ complex, the latter monoclonal antibody is said to bind weakly or not at all to tissues expressing αᵥβ₃ with the exception of osteoclasts and was suggested to be useful for in vivo therapy of bone disease. The former monoclonal antibody is suggested to have potential as a therapeutic agent in some cancers.

Ginsberg et al., U.S. Pat. No. 5,306,620 discloses antibodies that react with integrin so that the binding affinity of integrin for ligands is increased. As such these monoclonal antibodies are said to be useful for preventing metastasis by immobilizing melanoma tumors. Brown, U.S. Pat. No. 5,057,604 discloses the use of monoclonal antibodies to αᵥβ₃ integrins that inhibit RGD-mediated phagocytosis enhancement by binding to a receptor that recognizes RGD sequence containing proteins. Plow et al., U.S. Pat. No. 5,149,780 discloses a protein homologous to the RGD epitope of integrin β subunits and a monoclonal antibody that inhibits integrin-ligand binding by binding to the β₃ subunit. That action is said to be of use in therapies for adhesion-initiated human responses such as coagulation and some inflammatory responses.

Carron, U.S. Pat. No. 6,171,588, describes monoclonal antibodies which can be used in a method for blocking αᵥβ₃-mediated events such as cell adhesion, osteoclast-mediated bone resorption, restenosis, ocular neovascularization and growth of hemangiomas, as well as neoplastic cell or tumor growth and dissemination. Other uses described are antibody-mediated targeting and delivery of therapeutics for disrupting or killing αᵥβ₃ bearing neoplasms and tumor-related vascular beds. In addition, the inventive monoclonal antibodies can be used for visualization or imaging of αᵥβ₃ bearing neoplasms or tumor related vascular beds by NMR or immunoscintigraphy.

Examples of the targeted therapeutic approach have been described in various patent publications and scientific articles. International Patent Application WO 93/17715 describes antibodies carrying diagnostic or therapeutic agents targeted to the vasculature of solid tumor masses through recognition of tumor vasculature-associated antigens. International Patent Application WO 96/01653 and U.S. Pat. No. 5,877,289 describe methods and compositions for in vivo coagulation of tumor vasculature through the site-specific delivery of a coagulant using an antibody, while International Patent Application WO 98/31394 describes the use of Tissue Factor compositions for coagulation and tumor treatment. International Patent Application WO 93/18793 and U.S. Pat. Nos. 5,762,918 and 5,474,765 describe steroids linked to polyanionic polymers which bind to vascular endothelial cells. International Patent Application WO 91/07941 and U.S. Pat. No. 5,165,923 describe toxins, such as ricin A, bound to antibodies against tumor cells. U.S. Pat. Nos. 5,660,827, 5,776,427, 5,855,866, and 5,863,538 also disclose methods of treating tumor vasculature. International Patent Application WO 98/10795 and WO 99/13329 describe tumor homing molecules, which can be used to target drugs to tumors.

Angiogenesis-related Targets

The growth of new blood vessels from existing endothelium (angiogenesis) is tightly controlled in healthy adults by opposing effects of positive and negative regulators. Under certain pathological conditions, including proliferative retinopathies, rheumatoid arthritis, psoriasis and cancer, positive regulators prevail and angiogenesis contributes to disease progression (reviewed in Folkman (1995) Nature Med. 1:27-31). In cancer, the notion that angiogenesis represents the rate limiting step of tumor growth and metastasis (Folkman (1971) New Engl. J. Med. 285:1182-1186) is now supported by considerable experimental evidence (reviewed in Aznavoorian et al. (1993) Cancer 71:1368-1383; Fidler and Ellis (1994) Cell 79:185-188; Folkman (1990) J. Natl. Cancer Inst. 82:4-6). The quantity of blood vessels in tumor tissue is a strong negative prognostic indicator in breast cancer (Weidner et al. (1992) J. Natl. Cancer Inst. 84:1875-1887), prostate cancer (Weidner et al. (1993) Am. J. Pathol. 143:401-409), brain tumors (Li et al.(1994) Lancet 344:82-86), and melanoma (Foss et al.(1996) Cancer Res. 56:2900-2903).

VEGF Signaling in Angiogenesis

A number of angiogenic growth factors have been described to date among which vascular endothelial growth factor (VEGF) appears to play a key role as a positive regulator of physiological and pathological angiogenesis (reviewed in Brown et al. (1997) in Control of Angiogenesis (Goldberg and Rosen, eds.), Birkhauser, Basel, 233-269; Thomas (1996) J. Biol. Chem. 271:603-606; Neufeld et al. (1999) FASEB J. 13:9-22). VEGF is a secreted disulfide-linked homodimer that selectively stimulates endothelial cells to proliferate, migrate, and produce matrix-degrading enzymes (Conn et al. (1990) Proc. Natl. Acad. Sci. U.S.A. 87:1323-1327; Ferrara and Henzel (1989) Biochem. Biophys. Res. Commun. 161:851-858; Gospodarowicz et al. (1989) Proc. Natl. Acad. Sci. U.S.A. 86:7311-7315; Pepper et al. (1991) Biochem. Biophys. Res. Commun. 181:902-906; Unemori etal. (1992) J. Cell. Physiol. 153:557-562), all of which are processes required for the formation of new vessels. In addition to being the only known endothelial cell specific mitogen, VEGF is unique among angiogenic growth factors in its ability to induce a transient increase in blood vessel permeability to macromolecules (hence its original and alternative name, vascular permeability factor) (Dvorak et al.(1979) J. Immunol. 122:166-174; Senger et al.(1983) Science 219:983-985; Senger et al.(1986) Cancer Res. 46:5629-5632). Increased vascular permeability and the resulting deposition of plasma proteins in the extravascular space assists the new vessel formation by providing a provisional matrix for the migration of endothelial cells (Dvorak et al.(1995) Am. J. Pathol. 146:1029-1039). Hyperpermeability is indeed a characteristic feature of new vessels, including those associated with tumors (Dvorak et al.(1995) Am. J. Pathol. 146:1029-1039). Furthermore, compensatory angiogenesis induced by tissue hypoxia is now known to be mediated by VEGF (Levy et al.(1996) J. Biol. Chem. 271:2746-2753); Shweiki et al. (1992) Nature 359:843-845).

VEGF is produced and secreted in varying amounts by virtually all tumor cells (Brown et al. (1997) in Control of Angiogenesis (Goldberg and Rosen, eds.), Birkhauser, Basel:233-269). Direct evidence that VEGF and its receptors contribute to tumor growth was recently obtained by a demonstration that the growth of human tumor xenografts in nude mice could be inhibited by neutralizing antibodies to VEGF (Kim et al. (1993) Nature 362:841-844), by the expression of flk-1 in dominant-negative inhibition experiments (Millauer et al. (1996) Cancer Res. 56:1615-1620; Millauer et al. (1994) Nature 367:576-579), by low molecular weight inhibitors of the receptor tyrosine kinase domain of the VEGF receptor (Strawn et al. (1996) Cancer Res. 56:3540-3545), or by the expression of antisense sequence to VEGF mRNA (Saleh et al. (1996) Cancer Res. 56:393-401). Importantly, the incidence of tumor metastases was also found to be dramatically reduced by VEGF antagonists (Asano et al. (1995) Cancer Res. 55:5296-5301; Warren et al. (1995) J. Clin. Invest. 95:1789-1797; Claffey et al. (1996) Cancer Res. 56:172-181; Melnyk et al. (1996) Cancer Res. 56:921-924). Inhibitors of VEGF signaling may thus have broad clinical utility as anticancer agents. In addition to cancer, as noted above, other proliferative diseases characterized by excessive neovascularization such as psoriasis, age-related macular degeneration, diabetic retinopathy, osteoarthritis, and rheumatoid arthritis could be treated with antagonists of VEGF signaling.

VEGF occurs in several forms (VEGF-121, VEGF-145, VEGF-165, VEGF-189, VEGF-206) as a result of alternative splicing of the VEGF gene that consists of eight exons (Houck et al. (1991) Mol. Endocrin. 5:1806-1814; Tischer et al. (1991) J. Biol. Chem. 266:11947-11954; Poltorak et al. (1997) J. Biol. Chem. 272:7151-7158). The three smaller forms are diffusable, while the larger two forms remain predominantly localized to the cell membrane as a consequence of their high affinity for heparin. VEGF-165 and VEGF-145 also bind to heparin (as a consequence of containing basic exon 7- and exon 6-encoded domains, respectively), albeit with somewhat lower affinity compared with VEGF-189 (that contains both exons 6 and 7). VEGF-165 appears to be the most abundant form in most tissues (Houck et al. (1991) Mol. Endocrinol. 5:1806-1814; Carmeliet et al. (1999) Nature Med. 5:495-502). VEGF-121, the only alternatively spliced form that does not bind to heparin, appears to have a somewhat lower affinity for the receptors (Gitay-Goren et al. (1996) J. Biol. Chem. 271:5519-5523) as well as lower mitogenic potency (Keyt et al. (1996) J. Biol. Chem. 271:7788-7795).

VEGF Receptors

Biological effects of VEGF are mediated by two homologous tyrosine kinase receptors, Flt-1 (VEGFR1) and Flk-1/KDR (VEGFR2) whose expression is highly restricted to cells of endothelial origin (de Vries et al. (1992) Science 255:989-991; Millauer et al. (1993) Cell 72:835-846; Terman et al. (1991) Oncogene 6:519-524). Both receptors have an extracellular domain consisting of seven IgG-like domains, a transmembrane domain and an intracellular tyrosine kinase domain. The affinity of VEGFR1 for VEGF (K_{d}=1-20 pM) is higher compared to that of VEGFR2 (K_{d}=50-770 pM) (Brown et al. (1997) in Regulation of Angiogenesis, supra; de Vries et al. (1992) Science 255:989-991; Terman et al. (1992) Biochem. Biophys. Res. Commun. 187:1579-1586). In human umbilical cord endothelial cells (HUVECs) in 2-dimensional culture, VEGFR2 is by far the more abundant receptor (Brown et al. (1997) in Regulation of Angiogenesis, supra). In vivo, however, in quiescent endothelial cells, both receptors are expressed at low levels (Kremer et al. (1997) Cancer Res. 57:3852-3859; Barleon et al. (1997) Cancer Res. 57:5421-5425).

Both receptors are substantially upregulated when endothelial cells are activated by a variety of stimuli. Hypoxia, for example, induces an increase in expression of both VEGFR1 and VEGFR2 in endothelial cells (Tuder et al. (1995) J. Clin. Invest. 95:1798-1807; Gerber et al. (1997) J. Biol. Chem. 272:23659-23667; Brogi et al. (1996) J. Clin. Invest. 97:469-476; Kremer et al. (1997) Cancer Res. 57:3852-3859). For VEGFR1, hypoxia leads to both direct activation via the flt-1 promoter that contains the hypoxia-inducible-factor-1 (HIF-1) consensus binding site (Gerber et al. (1997) J. Biol. Chem., supra) and indirect activation via hypoxia-induced VEGF (Barleon et al. (1997) Cancer Res., supra). VEGF-induced upregulation of VEGFR1 is mediated by both VEGFR1 and VEGFR2 (Barleon et al. (1997) Cancer Res., supra). VEGFR2 is upregulated by VEGF (through VEGFR2, but not VEGFR1) (Kremer et al. (1997) Cancer Res., supra; Wilting et al. (1996) Dev. Biol. 176:76-85) and possibly by a yet unidentified factor in hypoxia-conditioned media from myoblasts (Brogi et al. (1996) J. Clin. Invest., supra). The expression of VEGFR2 in endothelial cells is also upregulated by bFGF and this accounts in part for the synergistic activation of endothelial cells by VEGF and bFGF (Pepper et al. (1998) Exp. Cell Res. 241:414-425). In addition, since both kdr and flt-1 promoters contain a cis-acting fluid shear-stress-responsive element, VEGFR1 and VEGFR2 expression may be sensitive to variations in blood flow (Tuder et al. (1995) J. Clin. Invest., supra).

Experiments using porcine aortic endothelial (PAE) cells transfected with the flt-1 or kdr receptor genes have suggested that VEGFR2 is the primary transducer in endothelial cells of VEGF-mediated signals related to changes in cell morphology and mitogenicity (Waltenberger et al. (1994) J. Biol. Chem. 269:26988-26995). In the same study, stimulation of flt-1-transfected PAE cells with VEGF did not appear to produce detectable changes. More recently, however, it was demonstrated that VEGF signaling through VEGFR1 induces migration of monocytes and upregulation of tissue factor expression in both endothelial cells and monocytes (Clauss et al. (1996) J. Biol. Chem. 271:17629-17634; Barleon et al. (1996) Blood 87:3336-3343). Based on the observation that the extracellular domain of VEGFR2 is retained on a cation exchange resin only in the presence of VEGFR1 and that the VEGFR2 retention is enhanced when both VEGFR1 and VEGF were present, Kendall et al. have concluded that the two receptors have some affinity for one another and that this interaction is stabilized by VEGF (Kendall et al. (1996) Biochem Biophys. Res. Commun. 226:324-328). When both receptors are expressed on cell surface, it appears likely that the VEGFR1/R2 heterodimer constitutes at least a fraction of the binding-competent VEGF receptor.

Tumor-associated lymphangiogenesis and metastasis has been linked to VEGFR3/Flt-4. Inhibition of lymphangiogenesis by a soluble form of VEGFR-3 has been observed in experiments with transgenic mice (Makinen et al. (2001) Nature Medicine, 7(2), 199-205). VEGF-C and VEGF-D, which bind to VEGFR-3, are involved in lyphangiogenesis and tumor metastasis (Stacker et al. Nature Medicine (2001), 7(2), 186-191; Skobe et al. Nature Medicine (2001), 7(2), 192-198).

Gene Deletion Studies of VEGF and VEGF Receptors

The functions of VEGFR1 and VEGFR2 have further been elucidated by targeted gene deletion studies. While deletion of either VEGFR1 or VEGFR2 results in embryonic lethality as a result of vascular abnormalities, there are important differences in the two phenotypes.

In mice deficient in VEGFR1, endothelial cells are formed but organize into distended and dilated vessels (Fong et al. (1995) Nature 376:66-70). Interestingly, mice that only lack the tyrosine kinase domain of VEGFR1 (and thus display the receptor on cell surfaces that is incapable of signaling) are viable, with the only detectable abnormality being the strongly suppressed macrophage migration in response to VEGF (Hiratsuka et al. (1998) Proc. Natl. Acad. Sci. 95:9349-9354). Since vascular abnormalities of VEGFR1 knockout mice are similar to those observed in transgenic mice that overexpress VEGF during development, it has been suggested that VEGFR1 is primarily a negative regulator of VEGF signaling, and that partial inhibition of VEGF signaling is essential for proper vessel development (Hiratsuka et al. (1998) Proc. Natl. Acad. Sci., supra). It is relevant to note in this context that VEGFR1 also exists as an alternatively spliced secreted extracellular domain that acts as a potent inhibitor of VEGF (Kendall et al. (1993) Proc. Natl. Acad. Sci., U.S.A. 90:10705-10709). The importance of tightly controlled VEGF signaling during development is further evidenced by the lethal phenotype of mice that lack only one allele of the VEGF gene (Carmeliet et al. (1996) Nature 380:435-439; Ferrara et al. (1996) Nature 380:439-442) and also of mice that only express the smallest isoform of VEGF (VEGF-120) (Carmeliet et al. (1999) Nature Med. 5:495-502). Thus, deviations on either side of precisely determined levels of VEGF signaling result in embryonic lethality.

Mice deficient in VEGFR2 lack both endothelial cells and hematopoietic cells, a more severe phenotype compared to that of VEGFR1 knockout, that results in embryonic lethality at day 8 (Shalaby et al. (1995) Nature 376:62-66). This is presumably a consequence of the fact that these two cell types arise from a common, VEGFR2-expressing precursor, the hemangioblast (Eichmann et al. (1997) Proc. Natl. Acad. Sci. 94:5141-5146).

Structural Requirements for Binding

Crystal structure of the receptor-binding domain of VEGF (residues 8-109) has recently been reported (Muller et al. (1997) Proc. Natl. Acad. Sci., U.S.A. 94:7192-7197; Muller et al. (1997) Structure 5:1325-1338). In the VEGF homodimer, the monomers are oriented in an antiparallel manner with two intersubunit disulfide bonds being formed between Cys51 from one subunit and Cys60 from the other. The three intrasubunit disulfide bonds are clustered in a characteristic cysteine knot motif (Sun et al. (1995) Annu. Rev. Biophys. Biomol. Struct. 24:269-291) also observed in PDGF and TGFP2. Despite low sequence homology (about 20%), PDGF and VEGF have very similar structures. Both proteins have an elongated shape in which each of the subunits consist primarily of four antiparallel β strands connected with three solvent accessible loops. In the homodimer, loops I and III from one subunit are adjacent to loop II from the other subunit. Alanine-scanning mutagenesis studies of VEGF have identified discrete regions that are important for high affinity binding to VEGFR1 and VEGFR2 (Keyt et al. (1996) J. Biol. Chem. 271:5638-5646; Muller et al. (1997) Proc. Natl. Acad. Sci., U.S.A. 94:7192-7197). Amino acid residues most critical for binding of VEGF to VEGFR1 are D63 and E64 in loop II. Residues most critical for binding of VEGF to VEGFR2 are R82-H86 encompassing loop III, I46 in loop I and E64 in loop II. Knowledge of the importance of these regions for receptor binding has been utilized to generate VEGF mutants in which only one side of the VEGF homodimer was rendered defective for receptor binding (Siemeister et al. (1998) Proc. Natl. Acad. Sci., U.S.A. 95:4625-4629; Fuh et al. (1998) J. Biol. Chem. 273:11197-11204). As expected, such monovalent VEGF mutants are inhibitors of VEGF-induced signaling since they are deficient in their ability to dimerize the receptors. Interestingly, avidity effects play a greater role in the binding of VEGF to VEGFR2 than to VEGFR1. The affinity of monomeric VEGFR1 for wild-type VEGF dimer is reduced only about 2-fold compared to that of dimeric VEGFR1 (IgG fusion construct) (Weismann et al. (1997) Cell 91:695-704). In contrast, the affinity of monomeric VEGFR2 for VEGF is reduced 100-fold compared to the dimeric VEGFR2 (Fuh et al (1998) J. Biol. Chem., supra). Comparing only the monomeric forms, VEGFR1 binds to VEGF with about 100-fold higher affinity compared to VEGFR2.

Domain deletion studies of the extracellular region of the VEGF receptors have shown that out of seven IgG-like domains, domains 2 and 3 of VEGFR1 (Davis-Smyth et al. (1996) EMBO J. 15:4919-4927; Barleon et al. (1997) J. Biol. Chem. 272:10382-10388) and VEGFR2 (Fuh et al. (1998) J. Biol. Chem. 273:11197-11204; Shinkai etal. (1998) J. Biol. Chem. 273:31283-31288) are essential for VEGF binding. The crystal structure of the complex between VEGF₈₋₁₀₉ with IgG domain 2 of VEGFR1 (that bind to VEGF with only 60-fold reduced affinity compared to the entire extracellular domain of the receptor) shows the receptor to be in contact with both subunits of VEGF₈₋₁₀₉ in an interaction dominated by hydrophobic contacts (Weismann et al. (1997) Cell, supra).

VEGF-165 Receptors

In addition to VEGFR1 and VEGFR2, receptors that only bind VEGF-165 and not VEGF-121 have been identified on endothelial cells and some tumor cells (Soker et al. (1996) J. Biol. Chem. 271:5761-5767; Soker et al. (1997) J. Biol. Chem. 272:31582-31588; Omura et al. (1997) J. Biol. Chem. 272:23317-23322). One such receptor unrelated in sequence to the tyrosine kinase receptors and with a short cytoplasmic domain, neuropilin-1, is also a receptor for semaphorins which play a role in neuronal chemorepulsion during development (Soker et al. (1998) Cell 92:735-745). Since the binding of VEGF-165 to neuropilin-1 involves the exon 7-encoded domain that is not required for the binding to VEGFR1 and VEGFR2, it has been suggested that neuropilin-1 serves as a co-receptor for VEGF-165. The presence of such receptors on endothelial cells may in part account for the enhanced mitogenic activity of VEGF-165 compared to VEGF-121. Consistent with this notion is the observation that the cardiovascular system of neuropilin-1 knockout mice does not develop normally, leading to embryonic lethality (Kitsukawa et al. (1997) Neuron 19:995-1005). The questions of what role VEGF may play in neuronal development and conversely, whether semaphorins have a role in vascular development and function, remain to be answered.

Receptor Binding Specificity of Various Forms of VEGF and Other Proteins in the VEGF Family

In addition to the alternatively spliced forms of VEGF, additional species can be generated by proteolytic processing. Plasmin cleaves VEGF-165 and VEGF-189 between residues Arg-110 and Ala-111 to generate VEGF-110 as the amino terminus fragment (Keyt et al. (1996) J. Biol. Chem., supra; Plouëet al. (1997) J. Biol. Chem. 272:13390-13396). Since it contains the receptor binding domain (supra), VEGF-110 binds to both VEGFR1 and VEGFR2. Like VEGF-121, VEGF-110 does not bind to heparin and its potency is lower compared to that of VEGF-165 (Keyt et al. (1996) J. Biol. Chem., supra). Interestingly, VEGF-189 can bind to VEGFR1, but not VEGFR2 and this renders it inactive as an endothelial cell mitogen (Houck et al. (1991) Mol. Endocrinol., supra; Plouët et al. (1997) J. Biol. Chem. 272, supra). VEGF-189 thus requires proteolytic processing either by plasmin or by urokinase-type plasminogen activator (that cleaves VEGF-189 in the exon 6-encoded domain to generate a 40 kDa fragment) to gain ability to bind to VEGFR2 (Plouët et al. (1997) J. Biol. Chem., supra).

Proteins with sequence homology to VEGF (also referred to as VEGF-A) have recently been described including placenta growth factor (PlGF: Park et al. (1994) J. Biol. Chem. 269:25646-25654), VEGF-B (Olofsson et al. (1996) Proc. Natl. Acad. Sci., U.S.A. 93:2576-2581), VEGF-C (Lee et al. (1996) Proc. Natl. Acad. Sci., U.S.A. 93:1988-1992; Joukov et al. (1996) EMBO J. 15:290-298), VEGF-D (Achen et al. (1998) Proc. Natl. Acad. Sci., U.S.A. 95:548-553) and VEGF-E (Ogawa et al. (1998) J. Biol. Chem. 273:31273-31282). In terms of receptor binding specificity, PlGF and VEGF-B can bind only to VEGFR1 with high affinity. VEGF-C and VEGF-D bind to VEGFR2 and another related tyrosine kinase, Flt-4 or VEGFR3. The expression of VEGFR3 appears to be confined to lymphatic endothelial cells. VEGF-E, a protein encoded in the genome of the Orf virus, binds only to VEGFR2 (Ogawa et al. (1998) J. Biol. Chem. 273:31273-31282). Some of these proteins including PlGF and VEGF-B can form heterodimers with VEGF (Cao et al. (1996) J. Biol. Chem. 271:3154-3162; DiSalvo et al. (1996) J. Biol. Chem. 270:7717-7723). The function of these VEGF-related molecules in physiological and pathological conditions remains to be precisely defined, however, it is clear that some redundancy of signaling mediated by VEGF receptors exists (Nicosia (1998) Am. J. Pathol. 153:11-16).

VEGF Receptors on Non-endothelial Cells

Although VEGFR1 and VEGFR2 are expressed predominantly on endothelial cells, they have also been detected on some non-endothelial cells. VEGFR1 is expressed on trophoblasts (Charnockjones et al. (1994) Biol. Reprod. 51:524-530), monocytes (Barleon et al. (1996) Blood, supra), hematopoietic stem cells and megakaryocytes/platelets (Katoh et al. Cancer Res. 55:5687-5692), renal mesangial cells (Takahashi et al. (1995) Biochem. Biophys. Res. Commun. 209:218-226) and pericytes (Yamagishi et al. (1999) Lab. Invest. 79:501-509). In monocytes, VEGFR1 is responsible for the VEGF-mediated induction of migration and tissue factor expression (Clauss et al. (1996) J. Biol. Chem., supra; Barleon et al. (1996) Blood, supra; Hiratsuka et al. (1998) Proc. Natl. Acad. Sci., supra). In pericytes, VEGFR1 may mediate the recently described ability of VEGF to act as a mitogen and chemotactic factor (Yamagishi et al. (1999) Lab. Invest., supra). The role of VEGFR1 in trophoblasts and mesangial cells remains to be elucidated. The expression of VEGFR2 has been detected on hematopoietic stem cells, megakaryocytes/platelets and retinal progenitor cells (Katoh et al. (1995) Cancer Res. 55:5687-5692; Yang et al. (1996) J. Neurosci. 16:6089-6099). VEGFR1 and VEGFR2 expression has also been reported on malignant cells including leukemia cells (Katoh et al. (1995) Cancer Res., supra) and melanoma cells (Gitay-Goren et al. (1993) Biochem. Biophys. Res. Commun. 190:702-709).

In Tabata, et al., Int. J Cancer 1999 82:737-42, antibodies are used to deliver radioactive isotopes to proliferating blood vessels. Ruoslahti & Rajotte, Annu. Rev. Immunol. (2000) 18:813-27; Ruoslahti, Adv. Cancer Res. 1999 76:1-20, review strategies for targeting therapeutic agents to angiogenic neovasculature, while Arap, et al., Science 1998 279:377-80 describe selection of peptides which target tumor blood vessels. Binetruy-Tournaire et al. report that peptide ATWLPPR binds to VEGFR-2 in-vitro, blocks the binding of VEGF to cell-displayed KDR, and inhibits angiogenesis in a rabbit cornea model (Binetruy-Tournaire et al., EMBO J. (2000) 19(7):1525-1533).

It should be noted that the typical arrangement used in such systems is to link the targeting entity to the therapeutic entity via a single bond or a relatively short chemical linker. Examples of such linkers include SMCC (succinimidyl 4-[N-maleimidomethyl]cyclohexane-1-carboxylate) or the linkers disclosed in U.S. Pat. No. 4,880,935, and oligopeptide spacers. Carbodiimides and N-hydroxysuccinimide reagents have been used to directly join therapeutic and targeting entities with the appropriate reactive chemical groups.

The use of cationic organic molecules to deliver heterologous genes in gene therapy procedures has been reported in the literature. Not all cationic compounds will complex with DNA and facilitate gene transfer. Currently, a primary strategy is routine screening of cationic molecules. The types of compounds which have been used in the past include cationic polymers such as polyethyleneamine, ethylene diamine cascade polymers, and polybrene. Proteins, such as polylysine with a net positive charge have also been used. The largest group of compounds, cationic lipids, includes DOTMA, DOTAP, DMRIE, DC-chol, and DOSPA. All of these agents have proven effective but suffer from potential problems such as toxicity and expense in the production of the agents. Cationic liposomes are currently the most popular system for gene transfection studies. Cationic liposomes serve two functions: protect DNA from degradation and increase the amount of DNA entering the cell. While the mechanisms describing how cationic liposomes function have not been fully delineated, such liposomes have proven useful in both in vitro and in vivo studies. However, these liposomes suffer from several important limitations. Such limitations include low transfection efficiencies, expense in production of the lipids, poor colloidal stability when complexed to DNA, and toxicity.

Although conjugates of targeting entities with therapeutic entities via relatively small linkers have attracted much attention, far less attention has been focused on using large particles as linkers. Typically, the linker functions simply to connect the therapeutic and targeting entities, and consideration of linker properties generally focuses on avoiding interference with the entities linked, for example, avoiding a linkage point in the antigen binding site of an immunoglobulin.

Large particulate assemblies of biologically compatible materials, such as liposomes, have been used as carriers for administration of drugs and paramagnetic contrast agents. U.S. Pat. Nos. 5,077,057 and 5,277,914 teach preparation of liposome or lipidic particle suspensions having particles of a defined size, particularly lipids soluble in an aprotic solvent, for delivery of drugs having poor aqueous solubility. U.S. Pat. No. 4,544,545 teaches phospholipid liposomes having an outer layer including a modified, cholesterol derivative to render the liposome more specific for a preselected organ. U.S. Pat. No. 5,213,804 teaches liposome compositions containing an entrapped agent, such as a drug, which are composed of vesicle-forming lipids and 1 to 20 mole percent of a vesicle-forming lipid derivatized with hydrophilic biocompatible polymer and sized to control its biodistribution and recirculatory half life. U.S. Pat. No. 5,246,707 teaches phospholipid coated microcrystalline particles of bioactive material to control the rate of release of entrapped water soluble biomolecules, such as proteins and polypeptides. U.S. Pat. No. 5,158,760 teaches liposome encapsulated radioactive labeled proteins, such as hemoglobin.

U.S. Pat. Nos. 5,512,294 and 6,090,408, and 6,132,764 (the contents of which are hereby incorporated by reference herein), describe the use of polymerized liposomes for various biological applications. One listed embodiment is to targeted polymerized liposomes which may be linked to or may encapsulate a therapeutic compound, (e.g., proteins, hormones or drugs) for directed delivery of a treatment agent to specific biological locations for localized treatment. Other publications describing liposomal compositions include U.S. Pat. Nos. 5,663,387, 5,494,803, and 5,466,467, to liposomes containing polymerized lipids for non-covalent immobilization of proteins and enzymes; Storrs et al., “Paramagnetic Polymerized Liposomes: Synthesis, Characterization, and Applications for Magnetic Resonance Imaging,” J. Am. Chem. Soc. (1995) 117(28):7301-7306; and Storrs et al., “Paramagnetic Polymerized Liposomes as New Recirculating MR Contrast Agents,” JMRI (1995) 5(6):719-724; Wu et al., “Metal-Chelate-Dendrimer-Antibody Constructs for Use in Radioimmunotherapy and Imaging,” Bioorganic and Medicinal Chemistry Letters (1994) 4(3):449-454 is a publication directed to dendrimer-based compounds.

The need for recirculation of therapeutic agents in the body, that is avoidance of rapid endocytosis by the reticuloendothelial system and avoidance of rapid filtration by the kidney, to provide sufficient concentration at a targeted site to afford necessary therapeutic effect has been recognized. Experience with magnetic resonance contrast agents has provided useful information regarding circulation lifetimes. Small molecules, such as gadolinium diethylenetriaminepentaacetic acid, tend to have limited circulation times due to rapid renal excretion while most liposomes, having diameters greater than 800 nm, are quickly cleared by the reticuloendothelial system. Attempts to solve these problems have involved use of macromolecular materials, such as gadolinium diethylenetriaminepentaacetic acid derived polysaccharides, polypeptides, and proteins. These agents have generally not demonstrated the versatility in chemical modification to provide for both long recirculation times and active targeting.

The association of liposomes with polymeric compounds in order to avoid rapid clearance in the liver, or for other stabilizing effects has been described. For example, Dadey, U.S. Pat. No. 5,935,599 described polymer-associated liposomes containing a liposome, and a polymer having a plurality of anionic moieties in a salt form. The polymer may be synthetic or naturally-occurring. The polymer-associated liposomes remain in the vascular system for an extended period of time.

Polysaccharides are one class of polymeric stabilizer. Calvo Salve, et al., U.S. Pat. No, 5,843,509 describe the stabilization of colloidal systems through the formation of lipid-polysaccharide complexes and development of a procedure for the preparation of colloidal systems involving a combination of two ingredients: a water soluble and positively charged polysaccharide and a negatively charged phospholipid. Stabilization occurs through the formation, at the interface, of an ionic complex: aminopolysaccharide-phospholipid. The polysaccharides utilized by Calvo Salve, et al., include chitin and chitosan.

Dextran is another polysaccharide whose stabilizing properties have been investigated. Cansell, et al., J. Biomed. Mater. Res. 1999, 44:140-48, report that dextran or functionalized dextran was hydrophobized with cholesterol, which anchors in the lipid bilayer of liposomes during liposome formation, resulting in a liposome coated with dextran. These liposomes interacted specifically with human endothelial cells in culture. In Letoumeur, et al., J. Controlled Release 2000, 65:83-91, the antiproliferative functionalized dextran-coated liposomes were used as a targeting agent for vascular smooth muscle cells. Ullman, et al. Proc. Nat. Acad. Sci 91:5426-30 (1994) and Ullman, et al., Clin. Chem. 42:1518-26 (1996) describe the coating of polystyrene beads with dextran and the attachment of ligands, nucleic acids, and proteins to the dextran-polystyrene complexes.

Dextran has also been used to coat metal nanoparticles, and such nanoparticles have been used primarily as imaging agents. For example, Moore, et al., Radiology 2000, 214:568-74, report that in a rodent model, long-circulating dextran-coated iron oxide nanoparticles were taken up preferentially by tumor cells, but also were taken up by tumor-associated macrophages and, to a much lesser extent, endothelial cells in the area of angiogenesis. Groman, et al., U.S. Pat. No. 4,770,183, describe 10-5000 Å superparamagnetic metal oxide particles for use as imaging agents. The particles may be coated with dextran or other suitable polymer to optimize both the uptake of the particles and the residence time in the target organ. A dextran-coated iron oxide particle injected into a patient's bloodstream, for example, localizes in the liver. Groman, et al., also report that dextran-coated particles can be preferentially absorbed by healthy cells, with less uptake into cancerous cells.

Lewis, et al., U.S. Pat. No. 5,055,288, describe the preparation and isolation of biodegradeable supraparamagnetic MR imaging agents for vascular compartments. The imaging agents are aggregates of individual biodegradeable superparamagnetic metal oxide crystals, which may be formed in the presence of macromolecular species, including dextran.

Kresse, et al., U.S. Pat. No. 6,048,515 report that iron-containing nanoparticles with a polymer coating, including dextran, may be further coated with a “targeting polymer” which targets the particle to a specific tissue. The resulting particles may be used for imaging or as therapeutics.

Utilization of liposomes as carriers of radionuclides for therapeutic applications has not been widely reported. One major hurdle in this area is the efficient labeling of liposomes with therapy nuclides. One strategy is described in Häfeli, et al., Nucl. Med. Biol. (1991) 18:449-54. In Häfeli, et al., liposomes with a 70 nm diameter were made by the detergent removal technique on a gel filtration column, and a radioactive Re complex was incorporated into the bilayer of the liposomes during liposome formation. The stablility of these radioactive liposomes was tested by dialysis, and a loss of 40% of the radioactivity identified as perrhenate was observed after 8 days. Addition of the antioxidant ascorbic acid diminished the loss to 20%. Häfeli, et al., suggest that liposomes carrying the lipophilic radioactive Re-complex can potentially be used in beta-radiotherapy.

Another report, Utkhede, et al., J. Liposome Res. (1994) 4:1049-1061, describes 90-Y entrapment into SUV's and PEG-coated liposomes via the cation ionophore A23187. After transport across the lipid bilayer, 90-Y was chelated in the vesicle interior by DTPA. No loading occurs at 40° C., and 89.2-95.9% loading occurs at 41-50° C. No in vivo biodistribution studies were reported, nor any dosimetry studies to assess the therapuetic potential of the liposomes.

Although various liposome systems have been presented that exhibit preferential tumor localization, very little work has been described investigating the possible therapeutic effects of liposome delivered particle-emitting radionuclides to tumors. Kosterelos, et al., J. Liposome Res. (1999) 9:407-24, reviewed the use of liposomes for imaging and therapy. The report states that “there has not been a single study in the literature utilizing liposomes as carriers of radionuclides for therapeutic applications” (although this is not strictly true) and further suggests that the success or failure of any radiotherapeutic modality will be critically dependent on its proper dosimetry assessment. In a more recent theoretical publication by Kostarelos & Emfietzoglou, Anticancer Res. (2000) 20:3339-45, dosimetry estimates for liposomes containing various isotopes were calculated from previously reported biodistribution data for liposome-isotope complexes. Multilamellar (MLV), small unilamellar (SUV) and sterically stabilized (GM1- and PEG-coated) liposomes were examined in combination with the particle emitting radionuclides 67-Cu, 188-Re and 211-At, 90-Y and 131-I. Regardless of radionuclide, the poorest values were obtained for the MLV liposomes. Sterically stabilized (GM-coated) liposomes are taken up by the muscle tumor tissue more readily than are SUVs. As a result, 211 -At and 188-Re deliver higher tumor doses when combined with the former, but 67-Cu, 90-Y and 131-I are more effective with the latter. Kostarelos & Emfietzoglou conclude that the importance of liposome size and steric barrier when designing effective radionuclide-carrier systems, as well as optimal matching between the radionuclide half-life and the time of maximum liposome accumulation ratio between tumor and normal tissue, are important considerations. A description of the use of ⁹⁰Y-liposome complexes for therapy was not provided in this theoretical report.

Bard, et al., Clin. Exp. Rheumatol. (1985) 3:237-42, have studied the effect of the intra-articular injection of lutetium-177 in chelator liposomes on the progress of an experimental arthritis in rabbits. The liposomes were prepared by combining 3-cholesteryl 6-[N′-iminobis(ethylenenitrilo)tetraacetic acid acid]hexyl ether (Chol-DTTA) with DSPC and a radioactive isotope, either 51-Cr or 177-Lu. The treatment of rheumatoid arthritis by radiosynovectomy has been restricted by the difficulty of preventing leakage of the radioisotope from the joint cavity. In this study, liposomes were prepared with 3-cholesteryl 6-[N′-iminobis(ethylenenitrilo)-tetraacetic acid]hexyl ether (Chol-DTTA) which can complex with a variety of beta-emitting radionuclides. In a previous study, Bard, et al., Clin. Exp. Rheumatol. (1983) 1:113-7, 51 -Cr was used as the radioisotope. The liposomes were injected into the knee joint cavity of rabbits with expertimentally induced arthritis. For the 51-Cr liposomes, greater than 99% of the radioactivity was retained in the joint after 24 hours, with 93% of the radioactivity associated with the synovium (the membrane that covers synovial joints and secretes synovial fluid, and lubricates the joints). In the case of 177-Lu, reported losses of radioactivity averages less than 1% over 47 days, and that low radiation dose resulted in very little synovitis with no damage to the knee cartilage.

The preliminary results of Kosterelos, et al. and Bard, et al., are encouraging, but in general, therapeutic applications of therapy radionuclides in conjunction with liposomes have been ignored. Accordingly, there remains a need for methods of preparation of stable liposomes suitable for delivery of therapeutic radionuclides in a variety of applications. Furthermore, there remains a need for methods of preparation of stable liposomes containing a targeting agent for the delivery of therapeutic radionuclides. There is a further need for methods of preparation of stable liposomes containing a targeting agent and an imaging agent along with a therapeutic isotope.

### SUMMARY OF THE INVENTION

The present invention provides lipid constructs comprising a linking carrier, a targeting entity, and optionally, a therapeutic entity. In preferred embodiments, the linking carrier is a polymerized liposome. Polymerized liposomes comprise polymerizable lipids of which 1,2-bis(10,12-tricosadiynoyl)-sn-glycero-3-phosphocholine and [PDA-PEG₃]₂-DTTA₃ are preferred. In preferred embodiments, the therapeutic entity is a metal ion, and even more preferably a radioactive metal ion such as Y-90, Bi-213, At-211, Cu-67, Sc-47, Ga-67, Rh-105, Pr-142, Nd-147, Pm-151, Sm-153, Ho-166, Gd-159, Tb-161, Eu-152, Er-171, Re-186, and Re-188, with 90-Y being particulary preferred. In preferred embodiments, the therapeutic metal ion is associated with the lipid construct via a lipid chelator. Preferred lipid chelators are N,N-bis[[[[(13′,15′-pentacosadiynamido-3,6-doxaoctyl)carbamoyl]methyl](carboxymethyl)amino]ethyl]glycine ([PDA-PEG₂]₂-DTTA₃), N,N-bis[[[[(13′,15′-pentacosadiynamido-3,6,9-trioxaundecyl)carbamoyl]methyl](carboxymethyl)amino]ethyl]glycine ([PDA-PEG₃]₂-DTTA₃), and 1,2-dimyristoyl-sn-glycero-3-phosphoethanolamidotriamine tetraacetic acid.

In some embodiments, the lipid chelator contains a diacetylene lipid or is a derivative of diethylenetriaminepentaacetic acid, a derivative of ethylaminediaminetetracetic acid, or a derivative of 1,4,7,10-tetraazacyclododecane-N,N′,N″,N′″-tetraacetic acid (DOTA). In other embodiments, the lipid chelator comprises an ionizable group such as carboxyl, phosphate, phosphonate, sulfate, sulfonate, or sulfinate. In still other embodiments, the lipid chelator comprises a single ionizable group, said single ionizable group generating a surface capable of binding an isotope or metal with a valency of +2 or greater, or +3 or greater.

In other preferred embodiments, the therapeutic entity is a chemotherapeutic agent or prodrug or toxin where the therapeutic entity is attached to the surface of the linking carrier. Alternatively, the therapeutic entity may be entrapped or encapsulated within the linking carrier.

The targeting entity in preferred embodiments is a small molecule ligand or a protein, such as an antibody. In preferred embodiments, the targeting entity targets the lipid construct to a cell surface target.

In some embodiments, the targeting entity is attached to the lipid construct through a group selected from the group consisting of amine, cyano, carboxylic acid, isothiocyanate, thiol, disulfide, α-halocarbonyl, α,β-unsaturated carbonyl or alkyl hydrazine. In other embodiments, the targeting entity is attached to the lipid construct by non-covalent means, with a biotin-avidin biotinylated antibody sandwich being preferred.

In particularly preferred embodiments, the targeting entity is an anti-VEGFR-2 antibody or an anti-integrin alpha v subunit antibody. In other embodiments, the targeting entity is an antibody that has a target selected from the group consisting of P-selectin, E-selectin, pleiotropin, chemokines and their receptors, cytokines and their receptors, G-protein coupled receptors, endosialin, endoglin, VEGF receptor, PDGF receptor, FGF or EGF receptor, the matrix metalloproteases, and prostate specific membrane antigen (PSMA).

In yet another embodiment, the targeting entity is a naturally occurring binding partner (i.e. a protein) of cell surface receptors including but not limited to P-selectin, E-selectin, pleiotropin, chemokines and their receptors, cytokines and their receptors, G-protein coupled receptors, endosialin, endoglin, VEGF receptor, PDGF receptor, FGF or EGF receptor, the matrix metalloproteases, and prostate specific membrane antigen (PSMA). In a further embodiment, the targeting entity is a derivative of the naturally occurring binding partner that contains sequences of the natural binding partner or a derivative that contains amino acids required for binding of the derivative to the cell surface receptor. In a particularly preferred embodiment, the binding partner is VEGF including the various forms of VEGF, and its derivatives and homologues. In another preferred embodiment, the binding partner is fibroblast growth factor (FGF) and its derivatives and homologues.

In preferred embodiments, the lipid construct further comprising a stabilizing agent, with dextran or aminodextran being preferred. In these embodiments, the targeting entity may be attached to the dextran derivative, which is attached to the lipid construct by covalent or non-covalent means. More, specifically, these embodiments include targeting agent-stabilizing entity-lipid construct-therapeutic entity complexes as well as targeting agent-stabilizing entity-lipid construct complexes where the therapeutic entity is an isotope or chemotherapeutic agent. Particularly preferred lipid constructs comprise a liposome or a polymerized liposome, an anti-VEGFR-2 antibody or an anti-alpha v integrin subunit antibody, and ⁹⁰Y.

Other particularly preferred lipid constructs comprise a liposome or polymerized liposome, an anti-VEGFR-2 antibody or an anti-alpha v integrin subunit antibody, ⁹⁰Y, and a dextran derivative.

Other preferred embodiments comprise a liposome or polymerized liposome, and an anti-VEGFR-2 antibody or an anti-alpha v integrin subunit antibody. Additional preferred embodiments comprise a liposome or polymerized liposome, and a ligand, peptide, or protein that binds to VEGFR-2, where the ligand, peptide, or protein is of natural or synthetic origin. Optionally, the ligand, peptide, or protein is attached to a dextran derivative that is attached to the lipid construct.

### BRIEF DESCRIPTION OF THE FIGURES

FIG. 1 shows mean normalized tumor volume vs. days post treatment for buffer, anti-VEGFR-2 antibody, anti-VEGFR-2 antibody-dexPV-Y90 complex, anti-VEGFR-2-antibody-dexPV complex, dexPV-Y90 complex, and anti-VEGFR-2-PV-Y90 complex where PV is a polymerized vesicle, dexPV is a dextran-coated polymerized vesicle, and Y90 is yttrium-90.

FIG. 2 shows normalized tumor volume vs. days post treatment for Ab-PV-Y90 complexes where Ab is an antibody. The antibodies are anti-VEGFR-2 and anti-alpha v integrin subunit.

FIG. 3 shows structures for chelator lipid [PDA-PEG₃]₂DTTA 1 and BisT-PC 2 (1,2-bis(10,12 tricosadiynoyl)-sn-glycero-3-phosphocholine).

FIG. 4 shows treatment of tumors in a mouse melanoma model with antibody-vesicle-yttrium-90 conjugates having cell surface targets.

### DETAILED DESCRIPTION OF THE PREFERRED EMBODIMENTS

This invention provides therapeutic agents which are comprised of a targeting entity, a linking carrier, a therapeutic or treatment entity (optionally), and methods for their use. Preferred targets are extracellular targets, such as the vascular endothelial growth factor (VEGF) receptors 1, 2, and 3 or an integrin or integrin subunit, particularly αᵥ subunit. In preferred embodiments, the targeting entity may be a peptide or assembly of peptides (i.e. an antibody or protein), polynucleotide (such as RNA or a modified RNA), or ligand (natural or synthetic) that binds to the target, such as targeting entities specific for VEGFR-1/Flt-1, VEGFR-2/Flk-1/KDR, and VEGFR-3/Flt4. In preferred embodiments, the therapeutic entity is a nucleic acid, drug, prodrug, or radioisotope. The linking carrier may be a colloid, micelle, dendrimer, or lipid construct that contains the therapeutic entity.

A lipid construct as used herein, is a structure containing lipids, phospholipids, or derivatives thereof comprising a variety of different structural arrangements which lipids are known to adopt in aqueous suspension. These structures include, but are not limited to, lipid bilayer vesicles, micelles, liposomes, emulsions, lipid ribbons or sheets, and may be complexed with a variety of drugs and components which are known to be pharmaceutically acceptable. In the preferred embodiment, the lipid construct is a polymerized liposome, also referred to as a polymerized vesicle. Common additional components in lipid constructs include cholesterol and alpha-tocopherol, among others. The lipid constructs may be used alone or in any combination which one skilled in the art would appreciate to provide the characteristics desired for a particular application. In addition, the technical aspects of lipid constructs and liposome formation are well known in the art and any of the methods commonly practiced in the field may be used with the present invention.

The therapeutic or treatment entity may be associated with the agent by covalent or non-covalent means. Preferably, the agent is further comprised of a stabilizing entity which imparts additional advantages to the therapeutic or imaging agent which are not provided by conventional stabilizing entities. The stabilizing entity may be associated with the agent by covalent or non-covalent means. As used herein, associated means attached to by covalent or noncovalent interactions. Once the stabilizing entity is associated with the agent, the agent may be referred to as a “stabilized agent,” or in a more specific fashion depending on the type of lipid construct used, i.e., “stabilized liposome,” or “stabilized polymerized liposome.” Targeting and/or therapeutic agents may be attached to the stabilizing entity.

Liposomes

As used herein, lipid refers to an agent exhibiting amphipathic characteristics causing it to spontaneously adopt an organized structure in water wherein the hydrophobic portion of the molecule is sequestered away from the aqueous phase. A lipid in the sense of this invention is any substance with characteristics similar to those of fats or fatty materials. As a rule, molecules of this type possess an extended apolar region and, in the majority of cases, also a water-soluble, polar, hydrophilic group, the so-called head-group. Phospholipids are lipids which are the primary constituents of cell membranes. Typical phospholipid hydrophilic groups include phosphatidylcholine and phosphatidylethanolamine moieties, while typical hydrophobic groups include a variety of saturated and unsaturated fatty acid moieties, including diacetylenes. Mixture of a phospholipid in water causes spontaneous organization of the phospholipid molecules into a variety of characteristic phases depending on the conditions used. These include bilayer structures in which the hydrophilic groups of the phospholipids interact at the exterior of the bilayer with water, while the hydrophobic groups interact with similar groups on adjacent molecules in the interior of the bilayer. Such bilayer structures can be quite stable and form the principal basis for cell membranes.

Lipid bilayer vesicle, as used herein, refers to a closed, fluid-filled microscopic sphere which is formed principally from individual molecules having polar (hydrophilic) and non-polar (lipophilic) portions. The hydrophilic portions may comprise phosphate, glycerophosphate, carboxy, sulfate, amino, hydroxy, choline and other polar groups and derivatives thereof. Examples of non-polar groups are saturated or unsaturated hydrocarbons such as alkyl, alkenyl or other lipid groups. Sterols (e.g., cholesterol) and other pharmaceutically acceptable components (including anti-oxidants like alpha-tocopherol) may also be included to improve vesicle stability or confer other desirable characteristics.

Additionally, lipids to which a targeting agent, such as a ligand, peptidomimetic, peptide, or other synthetic molecule, are attached, may be incorporated into liposomes by preparing mixtures of the targeting lipid or lipids with additional chemically distinct lipids. One or more chemically distinct targeting lipids may be mixed with other chemically distinct lipids.

Bilayer structures can also be formed into closed spherical shell-like structures which are called vesicles or liposomes. The liposomes employed in the present invention can be prepared using any one of a variety of conventional liposome preparatory techniques. As will be readily apparent to those skilled in the art, such conventional techniques include sonication, chelate dialysis, homogenization, solvent infusion coupled with extrusion, freeze-thaw extrusion, microemulsification, as well as others. These techniques, as well as others, are discussed, for example, in U.S. Pat. No. 4,728,578, U.K. Patent Application G.B. 2193095 A, U.S. Pat. Nos. 4,728,575, 4,737,323, International Application PCT/US85/01161, Mayer et al., Biochimica et Biophysica Acta, Vol. 858, pp. 161-168 (1986), Hope et al., Biochimica et Biophysica Acta, Vol. 812, pp. 55-65 (1985), U.S. Pat. No. 4,533,254, Mahew et al., Methods In Enzymology, Vol. 149, pp. 64-77 (1987), Mahew et al., Biochimica et Biophysica Acta, Vol. 75, pp. 169-174 (1984), and Cheng et al., Investigative Radiology, Vol. 22, pp. 47-55 (1987), and U.S. Ser. No. 428,339, filed Oct. 27, 1989. The disclosures of each of the foregoing patents, publications and patent applications are incorporated by reference herein, in their entirety. A solvent free system similar to that described in International Application PCT/US85/01161, or U.S. Ser. No. 428,339, filed Oct. 27, 1989, may be employed in preparing the liposome constructions. By following these procedures, one is able to prepare liposomes having encapsulated therein a gaseous precursor or a solid or liquid contrast enhancing agent.

The materials which may be utilized in preparing the liposomes of the present invention include any of the materials or combinations thereof known to those skilled in the art as suitable in liposome construction. The lipids used may be of either natural or synthetic origin. Such materials include, but are not limited to, lipids such as cholesterol, phosphatidylcholine, phosphatidylethanolamine, phosphatidylserine, phosphatidylglycerol, phosphatidic acid, phosphatidylinositol, lysolipids, fatty acids, sphingomyelin, glycosphingolipids, glucolipids, glycolipids, sulphatides, lipids with amide, ether, and ester-linked fatty acids, polymerizable lipids, and combinations thereof.

Additionally, the present invention includes lipid derivatives containing carboxyl, phosphate, phosphonate, sulfate, sulfonate, and sulfinate groups. As one skilled in the art will recognize, the liposomes may be synthesized in the absence or presence of incorporated glycolipid, complex carbohydrate, protein or synthetic polymer, using conventional procedures. The surface of a liposome may also be modified with a polymer, such as, for example, with polyethylene glycol (PEG), using procedures readily apparent to those skilled in the art. Lipids may contain functional surface groups for attachment to a metal, which provides for the chelation of radioactive isotopes or other materials that serve as the therapeutic entity. Any species of lipid may be used, with the sole proviso that the lipid or combination of lipids and associated materials incorporated within the lipid matrix should form a bilayer phase under physiologically relevant conditions. As one skilled in the art will recognize, the composition of the liposomes may be altered to modulate the biodistribution and clearance properties of the resulting liposomes.

The membrane bilayers in these structures typically encapsulate an aqueous volume, and form a permeability barrier between the encapsulated volume and the exterior solution. Lipids dispersed in aqueous solution spontaneously form bilayers with the hydrocarbon tails directed inward and the polar headgroups outward to interact with water. Simple agitation of the mixture usually produces multilamellar vesicles (MLVs), structures with many bilayers in an onion-like form having diameters of 1-10 μm (1000-10,000 nm). Sonication of these structures, or other methods known in the art, leads to formation of unilamellar vesicles (UVs) having an average diameter of about 30-300 nm. However, the range of 50 to 100 nm is considered to be optimal from the standpoint of, e.g., maximal circulation time in vivo. The actual equilibrium diameter is largely determined by the nature of the phospholipid used and the extent of incorporation of other lipids such as cholesterol. Standard methods for the formation of liposomes are known in the art, for example, methods for the commercial production of liposomes are described in U.S. Pat. No. 4,753,788 to Ronald C. Gamble and U.S. Pat. No. 4,935,171 to Kevin R. Bracken.

Either as MLVs or UVs, liposomes have proven valuable as vehicles for drug delivery in animals and in humans. Active drugs, including small hydrophilic molecules and polypeptides, can be trapped in the aqueous core of the liposome, while hydrophobic substances can be dissolved in the liposome membrane. Other molecules, such as DNA or RNA, may be attached to the outside of the liposome for gene therapy applications. The liposome constructs can be readily injected and form the basis for both sustained release and drug delivery to specific cell types, or parts of the body. MLVs, primarily because they are relatively large, are usually rapidly taken up by the reticuloendothelial system (the liver and spleen). The invention typically utilizes vesicles which remain in the circulatory system for hours and break down after internalization by the target cell through endocytosis. The invention may also utilize vesicles that bind to the target site and deliver a therapeutic agent to the desired site without internalization. In this case, the therapeutic agent may be a radioisotope that irradiates surrounding cells and cell layers. The therapeutic agent may also be a drug or pro-drug that is released while the invention is bound to the desired site. For these requirements the formulations preferably utilize UVs having an average diameter of less than 200 nm, more preferably less than 100 nm, and even more preferably about 60-80 nm.

Lipid constructs of the present invention also optionally include polymerizable lipids, which result in a lipid construct that is a polymerized liposome. Some preferred polymerizable lipids are [PDA-PEG₃]₂-DTTA, described as N,N-bis[[[[(13′,15′-pentacosadiynamido-3,6,9-trioxaundecyl)carbamoyl]methyl](carboxymethyl)amino]ethyl]glycine (compound 8a in JACS 1995, 117(28), 7301-7306) and 1,2-bis(10,12-tricosadiynoyl)-sn-glycero-3-phosphocholine (BisT-PC). Other polymerizable lipids include those disclosed in U.S. Pat. Nos. 5,512,294 and 6,132,764. The polymerized liposomes are generally prepared by polymerization of unsaturated monomeric phospholipids. These phospholipids may contain any unsaturated functional group, including polymerizable double or triple bonds, and may contain more than one polymerizable functional group. The functional groups irreversibly cross-link, or polymerize, when exposed to ultaviolet light or other radical, anionic or cationic, initiating species, while maintaining the distribution of functional groups at the surface of the liposome. Suitable monomeric phospholipids are known to those skilled in the art, and include, but are not limited to, phosphatidylcholines DODPC (1,2-di(2,4-Octadecadienoyl)-3-phosphatidylcholine), other phospholipids containing butadiene or hexatriene, diyne phospholipids, and lipids containing α,β-unsaturated ketones, esters, and aldehydes, see e.g., U.S. Pat. Nos. 4,485,045, 4,861,521.

The present invention also contemplates lipid constructs further comprising compounds, such as, for example, drugs or imaging agents, encapsulated or entrapped within the lipid constructs of the present invention. Methods for encapsulation of such entities are well known in the prior art.

Therapeutic Entities

The term “therapeutic entity” refers to any molecule, molecular assembly or macromolecule that has a therapeutic effect in a treated subject, where the treated subject is an animal, preferably a mammal, more preferably a human. The term “therapeutic effect” refers to an effect which reverses a disease state, arrests a disease state, slows the progression of a disease state, ameliorates a disease state, relieves symptoms of a disease state, or has other beneficial consequences for the treated subject. Therapeutic entities include, but are not limited to, drugs, such as doxorubicin, cisplatin, kinase inhibitors including nucleoside analogues and other chemotherapy agents; toxins such as ricin; radioactive isotopes; prodrugs (drugs which are introduced into the body in inactive form and which are activated in situ); and genes encoding proteins that exhibit cell toxicity. Radioisotopes useful as therapeutic entities are described in Kairemo, et al., Acta Oncol. 35:343-55 (1996), and include Y-90, I-123, I-125, I-131, Bi-213, At-211, Cu-67, Sc-47, Ga-67, Rh-105, Pr-142, Nd-147, Pm-151, Sm-153, Ho-166, Gd-159, Tb-161, Eu-152, Er-171, Re-186, and Re-188. The therapeutic or treatment entity may be associated with the lipid construct by covalent or non-covalent means. As used herein, associated means attached to the liposome by covalent or noncovalent interactions including ionic interactions and the formation of coordination complexes.

The present invention is also directed toward a therapeutic entity comprising the lipid constructs of the present invention. In a preferred embodiment, the therapeutic agent is a radionuclide. As used herein, a therapeutic radionuclide is a nuclide which undergoes spontaneous transformation (nuclear decay) with an energy transfer sufficient to impart cytotoxic amounts of radiant energy to nearby cells. In contrast, radionuclides useful for diagnosis emit radiation capable of penetrating tissue with minimal cell damage. Such radiation may be detected using a suitable scintigraphic imager. Therapeutic radionuclides of the present invention include, but are not limited to Y-90, I-123, I-125, I-131, Bi-213, At-211, Cu-67, Sc-47, Ga-67, Rh-105, Pr-142, Nd-147, Pm-151, Sm-153, Ho-166, Gd-159, Tb-161, Eu-152, Er-171, Re-186, and Re-188. Diagnostic or imaging nuclides of the present invention include, but are not limited to Tc-99m, In-111, Ga-67, Rh-105, I-123, Nd-147, Pm-151, Sm-153, Gd-159, Tb-161, Er-171, Re-186, Re-188, and Tl-201.

The present invention is also directed towards a therapeutic entity combined with an imaging agent where the therapeutic radionuclide resides in the same solution as that containing vesicles or liposomes and the imaging or diagnostic agent. Furthermore, the present invention is directed towards targeted lipid constructs where both yttrium-90 and indium-111 or a technetium isotope may be combined in the same solution containing the lipid constructs.

In a preferred embodiment, a therapeutic radionuclide is associated with the lipid construct by non-covalent means. In a particularly preferred embodiment, the therapeutic radionuclide is associated with a chelator that is chemically attached to a lipid in the lipid construct. In another particularly preferred embodiment, yttrium-90 is the therapeutic radionuclide, and [PDA-PEG₃]₂DTTA is the lipid chelator. Other lipid chelators which are preferred are 1,2-dimyristoyl-sn-glycero-3-phosphoethanolamidotriamine tetraacetic acid as defined above, lipid derivatives of diethylenetriaminepentaacetic acid including diethylenetriaminetetraacetic acids and diethylenetriaminetriacetic acids, derivatives of ethylaminediaminetetracetic acid, and derivatives of 1,4,7,10-tetraazacyclododecane-N,N′,N″,N′″-tetraacetic acid (DOTA). Additionally, other lipids containing ionizable groups including carboxyl, phosphates, phosphonates, sulfates, sulfonates, and sulfinates may be preferred. In another preferred embodiment, lipids containing a single ionizable group may self assemble to generate a surface capable of binding an isotope or metal with a valency of +2 or greater. In another preferred embodiment, lipids containing two ionizable groups may self assemble to generate a surface capable of binding an isotope or metal with a valency of +3 or greater. In both of these embodiments, a single metal ion would bind to 2 or more lipid head groups.

The present invention also provides methods for the preparation of lipid constructs of the present invention. In a preferred embodiment, the method comprises preparation of a lipid construct of the present invention, attachment of a targeting agent, and optionally, chelation of an isotope primarily to the surface of the liposome. The method of the present invention overcomes the deficiencies of the prior art by attaching a targeting agent to the liposome and by generating lipid constructs containing both a targeting agent and a therapeutic isotope. The therapeutic isotope may be attached to the targeting agent-lipid construct conjugate with high efficiency and without the need for the removal of unassociated isotope. Additionally, the therapeutic isotope of the present invention may be attached to the liposomes of the present invention without the use of extreme temperatures, e.g., at room temperature. Optionally, the targeting agent may be attached to a stabilizing entity that is attached to the lipid construct. The resulting targeting agent-lipid construct-isotope complex or targeting agent-stabilizing entity-lipid construct-isotope complex binds to a target in in-vivo or in the presence of serum or plasma in-vitro where the targeting agent binds to its target and the isotope is detected using the appropriate detection method and apparatus. Furthermore, administration of this therapeutic construct into a murine melanoma model results in the inhibition of tumor growth.

Targeting Entities

The term “targeting entity” refers to a molecule, macromolecule, or molecular assembly which binds specifically to a biological target. The targeting entity may be of natural, synthetic, or semi-synthetic origin. Examples of targeting entities include, but are not limited to, antibodies (including antibody fragments and other antibody-derived molecules which retain specific binding, such as Fab, F(ab′)2, Fv, and scFv derived from antibodies); receptor-binding ligands, such as hormones or other molecules that bind specifically to a receptor; cytokines, which are polypeptides that affect cell function and modulate interactions between cells associated with immune, inflammatory or hematopoietic responses; molecules that bind to enzymes, such as enzyme inhibitors; nucleic acid ligands or aptamers, and one or more members of a specific binding interaction such as biotin or iminobiotin and avidin or streptavidin. In one embodiment of the present invention, preferred targeting entities are molecules which specifically bind to targets including receptors, antigens, or other markers found on vascular cells. In another embodiment, preferred targeting entities bind to receptors, antigens or markers associated with cells comprising lymphatic vessels and lymphatic vessels found in diseased tissue, including the lymph nodes. More preferred are molecules which specifically bind to receptors, antigens or markers found on cells of angiogenic neovasculature or receptors, antigens or markers associated with tumor vasculature. The receptors, antigens or markers associated with tumor vasculature can be expressed on cells of vessels which penetrate or are located within the tumor, or which are confined to the inner or outer periphery of the tumor. In preferred embodiments, the targeting agent is targeted to VEGFR-1/Flt-1, VEGFR-2/Flk-1/KDR, and VEGFR-3/Flt4. In other preferred embodiments, the targeting agent is targeted to a cell-surface integrin. In particularly preferred embodiments, the targeting agent is an anti-VEGFR-2 antibody or anti-alpha v integrin subunit antibody.

In one embodiment, the invention takes advantage of pre-existing or induced leakage from the tumor vascular bed; in this embodiment, tumor cell antigens can also be directly targeted with agents that pass from the circulation system into the tumor interstitial volume.

In another embodiment, preferred targeting entities bind to receptors, antigens or markers associated with cells comprising lymphatic vessels and lymphatic vessels found in diseased tissue, including the lymph nodes. In a preferred embodiment, preferred targeting entities bind to cellular targets in the lymphatic system and lymph nodes associated with cancer and cancer metastasis.

In another embodiment of the present invention, preferred targeting entities are molecules including ligands, peptides, proteins, or nucleic acids which specifically bind to receptors, antigens, or markers on cells that circulate within the vasculature, such as malignant B cells, or cells expressing antigens as a result of viral infection.

Targeting entities attached to the lipid constructs, linking carriers, or stabilizing entities of the invention include, but are not limited to, small molecule ligands, such as carbohydrates, and compounds such as those disclosed in U.S. Pat. No. 5,792,783 (small molecule ligands are defined herein as organic molecules with a molecular weight of about 1000 daltons or less, which serve as ligands for a vascular target or vascular cell marker); proteins, such as antibodies and growth factors; peptides, such as RGD-containing peptides (e.g., those described in U.S. Pat. No. 5,866,540), bombesin or gastrin-releasing peptide, peptides selected by phage-display techniques such as those described in U.S. Pat. No. 5,403,484, and peptides designed de novo to be complementary to tumor-expressed receptors; antigenic determinants; or other receptor targeting groups. These head groups can be used to control the biodistribution, non-specific adhesion, and blood pool half life of the polymerized liposomes. For example, β-D-lactose has been attached on the surface to target the asialoglycoprotein (ASG) found in liver cells which are in contact with the circulating blood pool. Glycolipids can be derivatized for use as targeting entities, for example, by converting the commercially available lipid (DAGPE) or the pentadicosanoic acid deravitive N-(8′-amino-3′,6′-dioxaoctyl)-10,12-pentacosadiynamide (PDA-PEG amine) into its isocyanate followed by treatment with triethyleneglycol diamine spacer 1,8-diamino-3,6-dioxaoctane to produce the amine terminated thiocarbamate lipid which by treatment with the para-isothiocyanophenyl glycoside of the carbohydrate ligand produces the desired targeting glycolipids. This synthesis provides a water soluble flexible spacer molecule spaced between the lipid that will form the internal structure or core of the liposome and the ligand that binds to cell surface receptors, allowing the ligand to be readily accessible to the protein receptors on the cell surfaces. The carbohydrate ligands can be derived from reducing sugars or glycosides, such as para-nitrophenyl glycosides, a wide range of which are commercially available or easily constructed using chemical or enzymatic methods. Polymerized liposomes coated with carbohydrate ligands can be produced by mixing appropriate amounts of individual lipids followed by sonication, extrusion, polymerization if polymerizable lipids are used, and filtration as described above. Suitable carbohydrate derivatized liposomes have about 1 to about 30 mole percent of the targeting glycolipid and filler lipid, such as PDA, DAPC, DAPE, or other phosphocholine based lipid, with the balance being metal-chelated lipid or metal-chelating lipid. Other lipids may be included in the lipid construct to assure liposome formation and provide high contrast and/or recirculation.

In some embodiments, the targeting entity targets the therapeutic construct to a cell surface. Delivery of the therapeutic or imaging agent can occur through endocytosis of the liposomes. Such deliveries are known in the art. See, for example Mastrobattista, et al., Immunoliposomes for the Targeted Delivery of Antitumor Drugs, Adv. Drug Del. Rev. (1999) 40:103-27.

In a preferred embodiment, the targeting entity is attached to a carboxyl head group on the lipid. In another preferred embodiment, the targeting entity is attached to a maleimide or the alpha-methyl group of an acetamide, such as iodo- or bromoacetamides. In one embodiment, the attachment is by covalent means. In another embodiment, the attachment is by non-covalent means. For example, antibody targeting entities may be attached by a biotin-avidin biotinylated antibody sandwich, to allow a variety of commercially available biotinylated antibodies to be used on the coated liposome. Specific vasculature targeting agents of use in the invention include (but are not limited to) anti-VCAM-1 antibodies (VCAM=vascular cell adhesion molecule); anti-ICAM-1 antibodies (ICAM=intercellularadhesion molecule); anti-integrin antibodies (e.g., antibodies directed against αᵥβ₃ integrin such as LM609, described in International Patent Application WO 89/05155 and Cheresh et al. J. Biol. Chem. 262:17703-11 (1987), and Vitaxin, described in International Patent Application WO 9833919 and in Wu et al., Proc. Natl. Acad. Sci. USA 95(11):6037-42 (1998); and antibodies or other targeting molecules which specifically bind P- and E-selectins, pleiotropin and endosialin, endoglin, chemokine receptors, cytokine receptors, VEGF receptors, PDGF receptors, FGF and EGF receptors, matrix metalloproteases, G-protein coupled receptors, MMPs, and prostate specific membrane antigen (PSMA). Additionally, the targeting agent may target cell surface polypeptides, polysaccharides, and carbohydrates such as extracellular matrix proteins vitronectin, fibronectin, and laminin; heparin; Lewis x, and Lewis y.

In one embodiment of the invention, the vascular-targeted therapeutic agent is combined with an agent targeted directly towards tumor cells. This embodiment takes advantage of the fact that the neovasculature surrounding tumors is often highly permeable or “leaky,” allowing direct passage of materials from the bloodstream into the interstitial space surrounding the tumor. Alternatively, the vascular-targeted therapeutic agent itself can induce permeability in the tumor vasculature. For example, when the agent carries a radioactive therapeutic entity, upon binding to the vascular tissue and irradiating that tissue, cell death of the vascular epithelium may follow and the integrity of the vasculature may be compromised.

In a preferred embodiment, the invention provides a vascular targeted therapeutic agent that comprises an integrin targeting agent or a VEGFR targeting agent and a ⁹⁰Y therapeutic entity.

Accordingly, in one embodiment, the vascular-targeted therapeutic agent has two targeting entities: a targeting entity directed towards a vascular marker, and a targeting entity directed towards a tumor cell marker. In another embodiment, an antitumor agent is administered with the vascular-targeted therapy agent. The antitumor agent can be administered simultaneously with the vascular-targeted therapy agent, or subsequent to administration of the vascular-targeted therapy agent. In particular, when the vascular-targeted therapy agent is relied upon to compromise vascular integrity in the area of the tumor, administration of the antitumor agent is preferably done at the point of maximum damage to the tumor vasculature.

The antitumor agent can be a conventional antitumor therapy, such as cisplatin, antibodies directed against tumor markers, such as anti-Her2/neu antibodies (e.g., Herceptin), or tripartite agents, such as those described herein for vascular-targeted therapy agent, but targeted against the tumor cell rather than the vasculature. A summary of monoclonal antibodies directed against various tumor markers is given in Table I of U.S. Pat. No. 6,093,399, hereby incorporated by reference herein in its entirety. In general, when the vascular-targeted therapy agent compromises vascular integrity in the area of the tumor, the effectiveness of any drug which operates directly on the tumor cells can be enhanced. The size of the vesicles can be adjusted for the particular intended end use including, for example, diagnostic and/or therapeutic use. As the size of the linking carrier can be manipulated readily, the overall size of the vascular-targeted therapeutic agents can be adapted for optimum passage of the particles through the permeable (“leaky” ) vasculature at the site of pathology, as long as the agent retains sufficient size to maintain its desired properties (e.g., circulation lifetime, multivalency). Accordingly, the particles can be sized at 30, 50, 100, 150, 200, 250, 300 or 350 nm in size, as desired. In addition, the size of the particles can be chosen so as to permit a first administration of particles of a size that cannot pass through the permeable vasculature, followed by one or more additional administrations of particles of a size that can pass through the permeable vasculature. The size of the vesicles may preferably range from about 30 nanometers (nm) to about 400 nm in diameter, and all combinations and subcombinations of ranges therein. More preferably, the vesicles have diameters ranging from about 10 nm to about 500 nm, with diameters ranging from about 40 nm to about 120 nm being even more preferred. In connection with particular uses, for example, intravascular use, including magnetic resonance imaging of the vasculature, it may be preferred that the vesicles be no larger than about 500 nm in diameter, with smaller vesicles being preferred, for example, vesicles of no larger than about 100 nm in diameter. It is contemplated that these smaller vesicles may perfuse small vascular channels, such as the microvasculature, while at the same time providing enough space or room within the vascular channel to permit red blood cells to slide past the vesicles.

While one major focus of the invention is the use of vascular-targeted therapy agent against the vasculature of tumors in order to treat cancer, the agents of the invention can be used in any disease where neovascularization or other aberrant vascular growth accompanies or contributes to pathology. Diseases associated with neovascular growth include, but are not limited to, solid tumors; blood born tumors such as leukemias; tumor metastasis; benign tumors, for example hemangiomas, acoustic neuromas, neurofibromas, trachomas, and pyogenic granulomas; rheumatoid arthritis; psoriasis; chronic inflammation; ocular angiogenic diseases, for example, diabetic retinopathy, retinopathy of prematurity, macular degeneration, comeal graft rejection, neovascular glaucoma, retrolental fibroplasia, rubeosis; arteriovenous malformations; ischemic limb angiogenesis; Osler-Webber Syndrome; myocardial angiogenesis; plaque neovascularization; telangiectasia; hemophiliac joints; angiofibroma; and wound granulation. Diseases of excessive or abnormal stimulation of endothelial cells include, but are not limited to, intestinal adhesions, atherosclerosis, restenosis, scleroderma, and hypertrophic scars, i.e., keloids.

Differing administration vehicles, dosages, and routes of administration can be determined for optimal administration of the agents; for example, injection near the site of a tumor may be preferable for treating solid tumors. Therapy of these disease states can also take advantage of the permeability of the neovasulature at the site of the pathology, as discussed above, in order to specifically deliver the vascular-targeted therapeutic agents to the interestitial space at the site of pathology.

Linking Carriers

The term “linking carrier” refers to any entity which A) serves to link the therapeutic entity and the targeting entity, and B) confers additional advantageous properties to the vascular-targeted therapeutic agents other than merely keeping the therapeutic entity and the targeting entity in close proximity. Examples of these additional advantages include, but are not limited to: 1) multivalency, which is defined as the ability to attach either i) multiple therapeutic entities to the vascular-targeted therapeutic agents (i.e., several units of the same therapeutic entity, or one or more units of different therapeutic entities), which increases the effective “payload” of therapeutic entity delivered to the targeted site; ii) multiple targeting entities to the vascular-targeted therapeutic agents (i.e., one or more units of different therapeutic entities, or, preferably, several units of the same targeting entity); or iii) both items i) and ii) of this sentence; and 2) improved circulation lifetimes, which can include tuning the size and composition of the particle to achieve a desirable rate of clearance by the reticuloendothelial system. The effective payload of therapeutic entity is the number of therapeutic entities delivered to the target site per binding event of the agent to the target. The payload will depend on the particular therapeuticentity and target. In some cases the payload will be as little as about 10 molecules delivered per binding event of the agent. In the case of a metal ion, the payload can be about 10³ molecules delivered per binding event. It is contemplated that the payload can be as high as 10⁴ molecules delivered per binding event. The payload can vary between about 10 to about 10⁴ molecules per binding event.

Preferred linking carriers are biocompatible polymers (such as dextran), macromolecular assemblies of biocompatible components (such as liposomes), or multi-component linking carriers consisting of more than one biocompatible component (such as dextran-coated liposomes). Examples of linking carriers include, but are not limited to, liposomes, polymerized liposomes, other lipid vesicles, micelles, dendrimers, polyethylene glycol assemblies, capped polylysines, poly(hydroxybutyric acid), dextrans, biocompatible polymers and copolymers such as hyaluronic acids and acrylamides and derivatives thereof, and polystyrene particles and derivatives thereof. A preferred linking carrier is a polymerized liposome. Polymerized liposomes are described in U.S. Pat. No. 5,512,294. Another preferred linking carrier is a dendrimer.

The linking carrier can be coupled to the targeting entity and the therapeutic entity by a variety of methods, depending on the specific chemistry involved. The coupling can be covalent or non-covalent. A variety of methods suitable for coupling of the targeting entity and the therapeutic entity to the linking carrier can be found in Hermanson, “Bioconjugate Techniques” , Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the therapeutic or targeting entity, e.g. a biotin-avidin interaction.

Polymerized liposomes are self-assembled aggregates of lipid molecules which offer great versatility in particle size and surface chemistry. Polymerized liposomes are described in U.S. Pat. Nos. 5,512,294 and 6,132,764, incorporated by reference herein in their entirety. The hydrophobic tail groups of polymerizable lipids are derivatized with polymerizable groups, such as diacetylene groups, which irreversibly cross-link, or polymerize, when exposed to ultaviolet light or other radical, anionic or cationic, initiating species, while maintaining the distribution of functional groups at the surface of the liposome. The resulting polymerized liposome particle is stabilized against fusion with cell membranes or other liposomes and stabilized towards enzymatic degradation. The size of the polymerized liposomes can be controlled by extrusion or other methods known to those skilled in the art. Polymerized liposomes may be comprised of polymerizable lipids, but may also comprise saturated and non-alkyne, unsaturated lipids. The polymerized liposomes can be a mixture of lipids which provide different functional groups on the hydrophilic exposed surface. For example, some hydrophilic head groups can have functional surface groups, for example, biotin, amines, cyano, carboxylic acids, isothiocyanates, thiols, disulfides, α-halocarbonyl compounds, α,β-unsaturated carbonyl compounds and alkyl hydrazines. These groups can be used for attachment of targeting entities, such as antibodies, ligands, proteins, peptides, carbohydrates, vitamins, nucleic acids or combinations thereof for specific targeting and attachment to desired cell surface molecules, and for attachment of therapeutic entities, such as drugs or radioactive isotopes.

Other head groups may have an attached or encapsulated therapeutic entity, such as, for example, antibodies, peptidomimetics, and hormones and drugs for interaction with a biological site at or near the specific biological molecule to which the polymerized liposome particle attaches. Other hydrophilic head groups can have a functional surface group of diethylenetriamine pentaacetic acid, ethylenedinitrile tetraacetic acid, tetraazocyclododecane-1,4,7,10-tetraacetic acid (DOTA), porphoryin chelate and cyclohexane-1,2,-diamino-N,N′-diacetate for attachment to a metal, which provides for the chelation of radioactive isotopes or other materials that serve as the therapeutic entity. Examples of lipids with chelating head groups are provided in U.S. Pat. No. 5,512,294, incorporated by reference herein in its entirety.

Large numbers of therapeutic entities may be attached to one polymerized liposome that may also bear from one to about one thousand targeting entities for in vivo adherence to targeted surfaces. The improved binding conveyed by multiple targeting entities can also be utilized therapeutically to block cell adhesion to endothelial receptors in vivo. Blocking these receptors can be useful to control pathological processes, such as inflammation and control of metastatic cancer. For example, multi-valent sialyl Lewis X derivatized liposomes can be used to block neutrophil binding, and antibodies against VCAM-1 on polymerized liposomes can be used to block lymphocyte binding, e.g. T-cells.

The polymerized liposome particle can also contain groups to control nonspecific adhesion and reticuloendothelial system uptake. For example, PEGylation of liposomes has been shown to prolong circulation lifetimes; see International Patent Application WO 90/04384.

The component lipids of polymerized liposomes can be purified and characterized individually using standard, known techniques and then combined in controlled fashion to produce the final particle. The polymerized liposomes can be constructed to mimic native cell membranes or present functionality, such as ethylene glycol derivatives, that can reduce their potential immunogenicity. Additionally, the polymerized liposomes have a well defined bilayer structure that can be characterized by known physical techniques such as transmission electron microscopy and atomic force microscopy.

Stabilizing Entities

The agents of the present invention optionally contain a stabilizing entity. As used herein, “stabilizing entity” refers to a macromolecule or polymer, which may optionally contain chemical functionality for the association of the stabilizing entity to the surface of the vesicle, and/or for subsequent association of therapeutic entities or targeting agents. The polymer should be biocompatible. Polymers useful to stabilize the liposomes of the present invention may be of natural, semi-synthetic (modified natural) or synthetic origin. A number of stabilizing entities which may be employed in the present invention are available, including xanthan gum, acacia, agar, agarose, alginic acid, alginate, sodium alginate, carrageenan, gelatin, guar gum, tragacanth, locust bean, bassorin, karaya, gum arabic, pectin, casein, bentonite, unpurified bentonite, purified bentonite, bentonite magma, and colloidal bentonite.

Other natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrose, dextrin, glucose, polyglucose, polydextrose, pustulan, chitin, agarose, keratin, chondroitin, dermatan, hyaluronic acid, alginic acid, xanthin gum, starch and various other natural homopolyner or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Other suitable polymers include proteins, such as albumin, polyalginates, and polylactide-glycolide copolymers. cellulose, cellulose (microcrystalline), methylcellulose, hydroxyethylcellulose, hydroxypropylcellulose, hydroxypropylmethylcellulose, carboxymethylcellulose, and calcium carboxymethylcellulose.

Exemplary semi-synthetic polymers include carboxymethylcellulose, sodium carboxymethylcellulose, carboxymethylcellulose sodium 12, hydroxymethylcellulose, hydroxypropylmethylcellulose, methylcellulose, and methoxycellulose. Other semi-synthetic polymers suitable for use in the present invention include carboxydextran, aminodextran, dextran aldehyde, chitosan, and carboxymethyl chitosan.

Exemplary synthetic polymers include poly(ethylene imine) and derivatives, polyphosphazenes, hydroxyapatites, fluoroapatite polymers, polyethylenes (such as, for example, polyethylene glycol, the class of compounds referred to as Pluronics®, commercially available from BASF, (Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof, polysorbate, carbomer 934P, magnesium aluminum silicate, aluminum monostearate, polyethylene oxide, polyvinylalcohol, povidone, polyethylene glycol, and propylene glycol. Methods for the preparation of vesicles which employ polymers to stabilize vesicle compositions will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

In a preferred embodiment, the stabilizing entity is dextran. In another preferred embodiment, the stabilizing entity is a modified dextran, such as amino dextran. Without being bound by theory, it is believed that dextran may increase circulation times of liposomes in a manner similar to PEG. In other preferred embodiments, the following polymers and their derivatives are used. poly(galacturonic acid), poly(L-glutamic acid), poly(L-glutamic acid-L-tyrosine), poly[R)-3-hydroxybutyric acid], poly(inosinic acid potassium salt), poly(L-lysine), poly(acrylic acid), poly(ethanolsulfonic acid sodium salt), poly(methylhydrosiloxane), poly(vinyl alcohol), poly(vinylpolypyrrolidone), poly(vinylpyrrolidone), poly(glycolide), poly(lactide), poly(lactide-co-glycolide), and hyaluronic acid. In other preferred embodiments, copolymers including a monomer having at least one reactive site, and preferably multiple reactive sites, for the attachment of the copolymer to the vesicle or other molecule.

In some embodiments, the polymer may act as a hetero- or homobifunctional linking agent for the attachment of targeting agents, therapeutic entities, or chelators such as DTPA and its derivatives.

In one embodiment, the stabilizing entity is associated with the vesicle by covalent means. In another embodiment, the stabilizing entity is associated with the vesicle by non-covalent means. Covalent means for attaching the targeting entity with the liposome are known in the art and described in the EXAMPLES section.

Noncovalent means for attaching the targeting entity with the liposome include but are not limited to attachment via ionic, hydrogen-bonding interactions, including those mediated by water molecules or other solvents, hyrdophobic interactions, or any combination of these.

In a preferred embodiment, the liposome the stabilizing agent forms a coating on the liposome, polymerized liposome, or other linking carrier.

Application of Therapeutic Agents to Other Body Fluids and Tissues

Preferred routes of administration of the cell-surface targeted therapeutic agents of the present invention are by intravenous, interperitoneal, or subcutaneous injection including administration to veins or the lymphatic system. While the primary focus of the invention is on vascular-targeted agents, in principle, a targeted agent can be designed to focus on markers present in other fluids, body tissues, and body cavities, e.g. synovial fluid, ocular fluid, or spinal fluid. Thus, for example, an agent can be administered to spinal fluid, where an antibody targets a site of pathology accessible from the spinal fluid. Intrathecal delivery, that is, administration into the cerebrospinal fluid bathing the spinal cord and brain, may be appropriate for example, in the case of a target residing in the choroid plexus endothelium of the cerebral spinal fluid (CSF)-blood barrier.

As an example of another treatment route of administration is one in which the disease to be treated is rheumatoid arthritis. In this embodiment of the invention, the invention provides therapeutic agents to treat inflamed synovia of people afflicted with rheumatoid arthritis. This type of therapeutic agent is a radiation synovectomy agent. Individuals with rheumatoid arthritis experience destruction of the diarthroidal or synovial joints, which causes substantial pain and physical disability. The disease will involve the hands (metacarpophalangeal joints), elbows, wrists, ankles and shoulders for most of these patients, and over half will have affected knee joints. Untreated, the joint linings become increasingly inflamed resulting in pain, loss of motion and destruction of articular cartilage. Chemicals, surgery, and radiation have been used to attack and destroy or remove the inflamed synovium, all with drawbacks.

The concentration of the radiation synovectomy agent varies with the particular use, but a sufficient amount is present to provide satisfactory radiation synovectomy. For example, in radiation synovectomy of the hip, the concentration of the agent will generally be higher than when used for the radiation synovectomy of the wrist joints. The radiation synovectomy composition is administered so that preferably it remains substantially in the joint for 20 half-lifes of the isotope although shorter residence times are acceptable as long as the leakage of the radionuclide is small and the leaked radionuclide is rapidly cleared from the body.

The radiation synovectomy compositions may be used in the usual way for such procedures. For example, in the case of the treatment of a knee-joint, a sufficient amount of the radiation synovectomy composition to provide adequate radiation synovectomy is injected into the knee-joint. There are a number of different techniques which can be used and the appropriate technique varies on the joint being treated. An example for the knee joint can be found, for example, in Nuclear Medicine Therapy, J. C. Harbert, J. S. Robertson and K. D. Reid, 1987, Thieme Medical Publishers, pages 172-3.

Another route of administration is through ocular fluid. In the eye, the retina is a thin layer of light-sensitive tissue that lines the inside wall of the back of the eye. When light enters the eye, it is focused by the cornea and the lens onto the retina. The retina then transforms the light images into electrical impulses that are sent to the brain through the optic nerve.

The macula is a very small area of the retina responsible for central vision and color vision. The macula allows us to read, drive, and perform detailed work. Surrounding the macula is the peripheral retina which is responsible for side vision and night vision. Macular degeneration is damage or breakdown of the macula, underlying tissue, or adjacent tissue. Macular degeneration is the leading cause of decreased visual acuity and impairment of reading and fine “close-up” vision. Age-related macular degeneration (ARMD) is the most common cause of legal blindness in the elderly.

The most common form of macular degeneration is called “dry” or involutional macular degeneration and results from the thinning of vascular and other structural or nutritional tissues underlying the retina in the macular region. A more severe form is termed “wet” or exudative macular degeneration. In this form, blood vessels in the choroidal layer (a layer underneath the retina and providing nourishment to the retina) break through a thin protective layer between the two tissues. These blood vessels may grow abnormally directly beneath the retina in a rapid uncontrolled fashion, resulting in oozing, bleeding, or eventually scar tissue formation in the macula which leads to severe loss of central vision. This process is termed choroidal neovascularization (CNV).

CNV is a condition that has a poor prognosis; effective treatment using thermal laser photocoagulation relies upon lesion detection and resultant mapping of the borders. Angiography is used to detect leakage from the offending vessels but often CNV is larger than indicated by conventional angiograms since the vessels are large, have an ill-defined bed, protrude below into the retina and can associate with pigmented epithelium.

Neovascularization results in visual loss in other eye diseases including neovascular glaucoma, ocular histoplasmosis syndrome, myopia, diabetes, pterygium, and infectious and inflammatory diseases. In histoplasmosis syndrome, a series of events occur in the choroidal layer of the inside lining of the back of the eye resulting in localized inflammation of the choroid and consequent scarring with loss of function of the involved retina and production of a blind spot (scotoma). In some cases, the choroid layer is provoked to produce new blood vessels that are much more fragile than normal blood vessels. They have a tendency to bleed with additional scarring, and loss of function of the overlying retina. Diabetic retinopathy involves retinal rather than choroidal blood vessels resulting in hemorrhages, vascular irregularities, and whitish exudates. Retinal neovascularization may occur in the most severe forms. When the vasculature of the eye is targeted, it should be appreciated that targets may be present on either side of the vasculature.

Delivery of the agents of the present invention to the tissues of the eye can be in many forms, including intravenous, ophthalmic, and topical. For ophthalmic topical administration, the agents of the present invention can be prepared in the form of aqueous eye drops such as aqueous suspended eye drops, viscous eye drops, gel, aqueous solution, emulsion, ointment, and the like. Additives suitable for the preparation of such formulations are known to those skilled in the art. In the case of a sustained-release delivery system for the eye, the sustained-release delivery system may be placed under the eyelid, conjunctiva, sclera, retina, optic nerve sheath, or in an intraocular or intraorbitol location. Intravitreal delivery of agents to the eye is also contemplated. Such intravitreal delivery methods are known to those of skill in the art. The delivery may include delivery via a device, such as that described in U.S. Pat. No. 6,251,090 to Avery.

Therapeutic Compositions

The present invention is also directed toward therapeutic compositions comprising the therapeutic agents of the present invention. Compositions of the present invention can also include other components such as a pharmaceutically acceptable excipient, an adjuvant, and/or a carrier. For example, compositions of the present invention can be formulated in an excipient that the animal to be treated can tolerate. Examples of such excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel™, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

One embodiment of the present invention is a controlled release formulation that is capable of slowly releasing a composition of the present invention into an animal. As used herein a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., biocrodible).

Generally, the therapeutic agents used in the invention are administered to an animal in an effective amount. Generally, an effective amount is an amount effective to either (1) reduce the symptoms of the disease sought to be treated or (2) induce a pharmacological change relevant to treating the disease sought to be treated. For cancer, an effective amount includes an amount effective to: reduce the size of a tumor; slow the growth of a tumor; prevent or inhibit metastases; or increase the life expectancy of the affected animal.

Therapeutically effective amounts of the therapeutic agents can be any amount or doses sufficient to bring about the desired anti-tumor effect and depend, in part, on the condition, type and location of the cancer, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compostions of the present invention. The method comprises administering the therapeutic agent to a subject in need of such administration.

The therapeutic agents of the instant invention can be administered by any suitable means, including, for example, parenteral, topical, oral or local administration, such as intradermally, by injection, or by aerosol. In the preferred embodiment of the invention, the agent is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic reagent of the present invention can be administered to any animal, preferably to mammals, and more preferably to humans.

In a further embodiment, the therapeutic agents of the present invention are useful for gene therapy. As used herein, the phrase “gene therapy” refers to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or (poly) peptide of therapeutic value. In a specific embodiment, the subject invention utilizes a class of lipid molecules for use in non-viral gene therapy which can complex with nucleic acids as described in Hughes, et al., U.S. Pat. No. 6,169,078, incorporated by reference herein in its entirety, in which a disulfide linker is provided between a polar head group and a lipophilic tail group of a lipid.

These therapeutic compounds of the present invention effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

Cationic lipid-polyanionic macromolecule aggregates may be formed by a variety of methods known in the art. Representative methods are disclosed by Felgner et al., supra; Eppstein et al. supra; Behr et al. supra; Bangham, A. et al. M. Mol. Biol. 23:238, 1965; Olson, F. et al. Biochim. Biophys. Acta 557:9, 1979; Szoka, F. et: al. Proc. Natl. Acad. Sci. 75: 4194, 1978; Mayhew, E. et al. Biochim. Biophys. Acta 775:169, 1984; Kim, S. et al. Biochim. Biophys. Acta 728:339, 1983; and Fukunaga, M. et al. Endocrinol. 115:757, 1984. In general aggregates may be formed by preparing lipid particles consisting of either (1) a cationic lipid or (2) a cationic lipid mixed with a colipid, followed by adding a polyanionic macromolecule to the lipid particles at about room temperature (about 18 to 26° C.). In general, conditions are chosen that are not conducive to deprotection of protected groups. In one embodiment, the mixture is then allowed to form an aggregate over a period of about 10 minutes to about 20 hours, with about 15 to 60 minutes most conveniently used. Other time periods may be appropriate for specific lipid types. The complexes may be formed over a longer period, but additional enhancement of transfection efficiency will not usually be gained by a longer period of complexing.

The compounds and methods of the subject invention can be used to intracellularly deliver a desired molecule, such as, for example, a polynucleotide, to a target cell. The desired polynucleotide can be composed of DNA or RNA or analogs thereof. The desired polynucleotides delivered using the present invention can be composed of nucleotide sequences that provide different functions or activities, such as nucleotides that have a regulatory function, e.g., promoter sequences, or that encode a polypeptide. The desired polynucleotide can also provide nucleotide sequences that are antisense to other nucleotide sequences in the cell. For example, the desired polynucleotide when transcribed in the cell can provide a polynucleotide that has a sequence that is antisense to other nucleotide sequences in the cell. The antisense sequences can hybridize to the sense strand sequences in the cell. Polynucleotides that provide antisense sequences can be readily prepared by the ordinarily skilled artisan. The desired polynucleotide delivered into the cell can also comprise a nucleotide sequence that is capable of forming a triplex complex with double-stranded DNA in the cell. The desired polynucleotide delivered into the cell can also be capable of other normal functions of polynucleotides; for example the polynucleotide could be a catalytic polynucleotide, e.g., ribozyme, or an siRNA.

This invention also provides a method of diagnosing abnormal pathology in vivo comprising, introducing a plurality of image-enhancing polymerized particles targeted to a molecular target involved in the abnormal pathology into a bodily fluid contacting the abnormal pathology, the targeting image enhancing polymerized particles attaching to a molecule involved in the abnormal pathology, and imaging in vivo the targeting image enhancing polymerized particles attached to molecules involved in the abnormal pathology.

Diagnostics

Antibody-conjugated liposomes of this invention achieve in vitro and in vivo targeting of specific molecules associated with specific body tissues and specific molecules associated with specific bodily functions and pathologies to provide sufficient signal enhancement for detection by imaging methods such as magnetic resonance imaging or nuclear scintigraphy. Such in vivo imaging of various disease or developmentally associated molecules permits following the relationship of these molecules to disease progression, their time course of progression, and their response to pharmacologic interventions. Characterization of these responses in individual animals simplifies assessment of the interventions, since expression and regression of the target can be confirmed as it relates to disease outcomes. As a diagnostic tool, this technique detects disease at early stages, thereby enabling more effective treatment. The liposomes of this invention are suitable for combination of imaging and delivery of drugs for therapeutic treatments. Various agents can be encapsulated or attached to the surface of liposomes for delivery to specific sites in vivo. By using target-specific drug/liposomes of this invention, the drug delivery can be simultaneously visualized by magnetic resonance imaging.

In one embodiment, the site-specific liposome having attached monoclonal antibodies for specific receptor targeting may be used to visualize abnormal pathology related to solid tumors, inflammation, rheumatoid arthritis, and osteoporosis using cell surface markers including the integrins, VEGF receptors, PDGF receptors, matrix metalloproteases, selecting, PSMA, endosialin, G-protein coupled receptors, and endoglin.

The present invention further provides methods and reagents for diagnostic purposes. Diagnostic assays contemplated by the present invention include, but are not limited to, receptor-binding assays, antibody assays, immunohistochemical assays, flow cytometry assays, genomics and nucleic acid detection assays. High-throughput screening arrays and assays are also contemplated.

This invention provides various methods for in vitro assays. For example, antibody-conjugated polymerized liposomes, according to this invention, provide an ultra-sensitive diagnostic assay for specific antigens in solution. Polymerized liposomes of this invention having a chelator head group chelated to spectroscopically distinct ions provide high sensitivity for assays involving protein-protein, ligand-protein, drug-protein, nucleic-acid protein, and nucleic acid-nucleic acid interactions. Polymerized liposomes of this invention having a fluorophore head group provide a method for detection of glycoproteins on cell surfaces.

Liposomes useful in diagnostic assays are described in U.S. Pat. No. 6,090,408, entitled “Use of Polymerized Lipid Diagnostic Agents,” and U.S. Pat. No. 6,132,764, entitled “Targeted Polymerized Liposome Diagnostic and Treatment Agents,” each incorporated by reference herein in its entirety.

In one embodiment of this invention, a targeting polymerized liposome particle comprises: an assembly of a plurality of liposome forming lipids each having an active hydrophilic head group linked by a bifunctional linker portion to the liposome forming lipid, and a hydrophobic tail group having a polymerizable functional group polymerized with a polymerizable functional group of an adjacent hydrophobic tail group of one of the plurality of liposome forming lipids, at least a portion of the hydrophilic head groups having an attached targeting active agent for attachment to a specific biological molecule. In another embodiment, the targeting polymerized liposome particle has a second portion of the hydrophilic head groups with functional surface groups attached to an image contrast enhancement agent to form a targeting image enhancing polymerized liposome particle. In yet another embodiment, a portion of the hydrophilic head groups have functional surface groups attached to or encapsulating a treatment agent for interaction with a biological site at or near the specific biological molecule to which the particle attaches, forming a targeting delivery polymerized liposome particle or a targeting image enhancing delivery polymerized liposome particle.

In a further embodiment, a treatment agent is encapsulated in the interior of the polymerized liposome or entrapped among the hydrophobic tails of the lipids.

This invention further provides a method of assaying abnormal pathology in vitro comprising, introducing a plurality of liposomes of the present invention to a molecule involved in the abnormal pathology into a fluid contacting the abnormal pathology, the targeting polymerized liposome particles attaching to a molecule involved in the abnormal pathology, and detecting in vitro the targeting polymerized liposome particles attached to molecules involved in the abnormal pathology.

Exemplary Targeted Lipid Constucts.

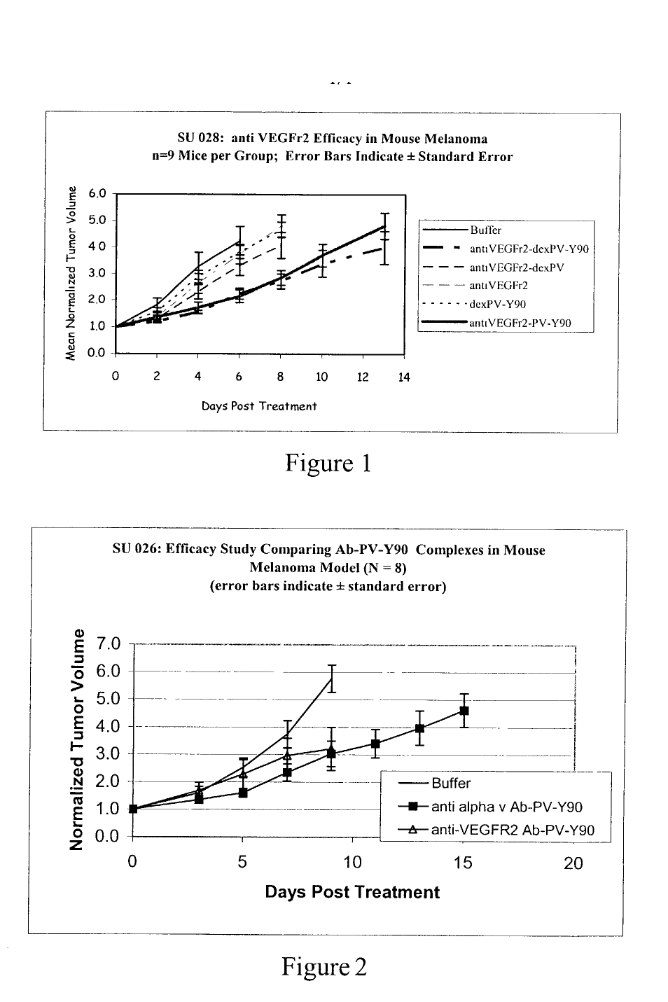

The targeted lipid constructs of the present invention have been shown to inhibit tumor growth in a mouse melanoma model. An anti-mouse VEGFR-2 antibody-dextran-polymerized vesicle-⁹⁰Y complex (Ab-dexPV-⁹⁰Y) and an identical complex that does not contain dextran were administered in accordance with EXAMPLE 5, and inhibit tumor growth as shown in FIG. 1. Control experiments with the anti-VEGFR-2 antibody-dexPV, dexPV-⁹⁰Y, and anti-VEGFR-2 antibody at the same concentration as that used for the Ab-PV complex do not significantly inhibit tumor growth relative to buffer. An anti-alpha v integrin subunit antibody-polymerized vesicle conjugate also inhibits tumor growth as shown in FIG. 2.

The following specific examples are set forth in detail to illustrate the invention and should not be considered to limit the invention in any way.

### EXAMPLES

### Example 1

Determination of Percent ⁹⁰Y Bound to Vesicles.

To 100 μL of the metal binding vesicles described in Examples 2 and 4 at 0.1-50 mg/mL approximately 100-250 μCi of ⁹⁰Y-C1₃ (Dupont NEN or Nordion) was added, mixed using a vortex mixer, and incubated at room temperature for 30 minutes. In duplicate, the percent ⁹⁰Y bound to the therapeutic vesicle was determined by adding 100 μL of the ⁹⁰Y-vesicle complex to a 100 k MWCO Nanosep™ (Pall Filtron) filter. The filter assembly was spun in a microfuge at 4000 rpm for 1 hr or until all of the solution has passed through the filter. The “total ⁹⁰Y” in the assembly was determined with the Capintec CRC-15R dosimeter and record the results. The filter portion of the assembly was removed and discarded. Using the dosimeter, the remaining part of the assembly containing the “unbound ⁹⁰Y” that passed through the filter was counted and the results recorded. “Bound ⁹⁰Y” was determined by subtracting the “unbound ⁹⁰Y” from the “total ⁹⁰Y” and the results recorded. Percent ⁹⁰Y bound was determined by dividing the “bound ⁹⁰Y” by the “total ⁹⁰Y” and multiplying by 100. ⁹⁰Y binding was greater than 95%.

### Example 2

Procedures for Coating Polymerized Vesicles (PVs) with Aminodextran and Coupling to Antibodies.

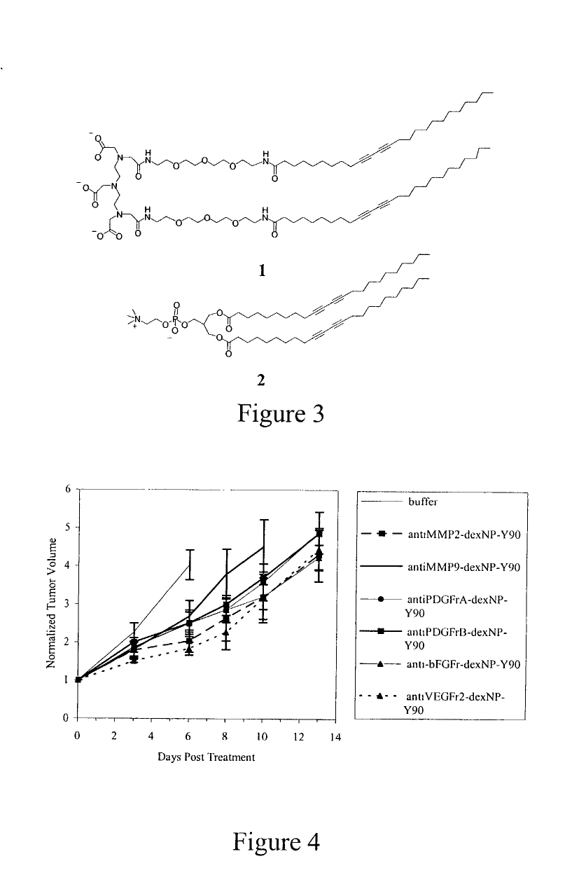

A. Coating the vesicles: PVs (polymerized vesicles composed of 95 mol % BisT-PC (1,2-bis(10,12-tricosadiynoyl)-sn-glycero-3-phosphocholine, compound 2, FIG. 3), 5 mol % [PDA-PEG3]-DTPA (compound 1, FIG. 3) 10 ml, 250 mg) were added dropwise to aminodextran (amine modified 10,000 MW dextran, Molecular Probes, product D-1860, 3.7 moles of amine/mole dextran, 500 mg) in 5 ml of 50 mM HEPES, pH 8 (N-[2-hydroxyethyl]piperazine-N′-[ethanesulfonic acid], Research Organics product 6003H) while stirring magnetically. EDAC (Aldrich 16146-2, ethyldimethylaminodipropyl carbodimimide HCl salt, 6 mg) in 200 μl water was added dropwise to the coating mixture while stirring. The mixture was left stirring at room temperature overnight. The clear reaction mixture was purified by size exclusion chromatography on a Sepharose CL 4B column (2.5×30 cm, Amersham Pharmacia Biotech AB product 17-0150-01) equilibrated with 10 mM HEPES, 200 mM NaCl pH 7.4. When the coated PVs began to elute 4 ml fractions were collected. The peak fractions (2 thru 6) were pooled and filtered through a 0.45μ filter (Nalgene 25 mm syringe filter, product 190-2545) followed by a 0.2μ filter (Nalgene 25 mm syringe filter, product 190-2520). The concentration of coated PV was determined by drying a sample to constant weight in an oven maintained at 90° C. The weight percent of aminodextran coating the PVs was determined by the anthrone method (T. A Scott and E. H. Melvin, Analytical Chemistry, Vol 25, No. 11, p. 1656, 1953).

B. Succinylation of aminodextran coat: Aminodextran-coated PVs (15 ml, 465 mg) in 10 mM HEPES pH 7.4 were diluted with an equal volume of 200 mM HEPES and the pH adjusted to 8 with 1 N NaOH. Succinic anhydride (Aldrich product 23,969-0, 278 mg) was dissolved in 1 ml DMSO (dimethyl sulfoxide (Aldrich product 27685-5) and 100 μL aliquots added to the PV suspension with rapid stirring. The pH was monitored and adjusted as necessary to maintain the pH between 7.5 and 8 by addition of 1 N NaOH. After the final addition of succinic anhydride the mixture was stirred for 1 hour at room temperature and then transferred to dialysis cassettes and dialyzed against 10 mM HEPES pH 7.4.

C. Coupling of antibody to succinamidodextran-PVs: Coated and succinylated PVs (succinamidodextran-PVs, 20 ml, 192 mg) and antibody (rat anti-mouse VEGFR-2 antibody, eBioscience cat# 14-5821 or rat anti-mouse alpha v subunit (CD51) antibody, PharMingen cat # 550023) were rapidly mixed while vortexing. EDAC (4 mg) in 400 μl water was added with vortexing and the mixture left at room temperature overnight. Other antibodies coupled to vesicles include anti-MMP-2, anti-MMP-9, anti-PDGF receptor, and anti-FGF receptor antibodies. The coupling reaction mixture was made 200 mM in NaCl to dissociate passively bound antibody from the surface of the PVs. After stirring at room temperature for 1 hour the mixture was purified by size exclusion chromatography on a column of Sepharose CL 4B equilibrated with 10 mM HEPES, 200 mM NaCl pH 7.4. Fractions (4 ml) were collected and assayed for antibody using commercially available reagents. No free unbound antibody was detected in the column fractions. PV containing fractions were pooled and dialyzed into 50 mM histidine, 5 mM citrate pH 7.4.

### Example 3

Test Material Processing

Test material will be labeled with 90-Y at the testing facility according to standard procedures. Briefly, yttrium-90 (90-Y) chloride solution will be diluted to a working concentration and a calculated volume containing a calculated amount of 90-Y in mCi will be added to test material. The solution will be mixed and the %90-Y bound to the vesicle will be determined for quality control as described above in Example 1.

### Example 4

Preparation of Vesicles

Liposomes were prepared from lipids in FIG. 3 as described in the following example: To a 100 mL round bottom flask was added 11 mL (220 mg, 240 μmol) of 1,2-bis(10,12-tricosadiynoyl)-sn-glycero-3-phosphocholine 2 lipid at 20 mg/mL chloroform and 3 mL (15 mg, 11 μmol) DTTA lipid 1 (compound 8, JACS (1995) 117(28), 7301-7306) at 5 mg/mL chloroform. The chloroform was removed at ≈60° C. by rotary evaporation. Water (10 mL) was added ant the solution was frozen on a dry ice/acetone mixture until solid. The pH was adjusted to 8 by adding 20 μL aliquots of 0.5 M NaOH. The freeze thaw process was repeated three times or until a translucent solution was obtained. This solution was passed through a 30 nm polycarbonate filter in a thermal barrel extruder (Lipex Biomembranes, Inc.) heated at 80° C. and pressurized with argon to 750 PSI. Vesicles were typically 60-65 nm as determined by dynamic light scattering (Brookhaven Instruments). Polymerization of diacetylene containing lipids was achieved by cooling the vesicles to 2-4° C. in a 10×1 polystyrene dish (VWR) and irradiating with UV light using a hand-held UV illuminator at approximately 3.8 mW/cm². The optical density at 500 nm for the orange vesicles was approximately 0.4 AU at 1 mg/mL of vesicle in water. Yellow vesicles were prepared by polymerization at 12° C. and the optical density was 1 AU at 1 mg/mL vesicle in water.

### EXAMPLE 5

Treatment of Solid Tumors in a Mouse Melanoma Model

1.1. Tumor Implantation

1.1.1. Cell Culture

The K1735-M2 (Li et al, Invasion Metastasis (1998), 18, 1-14) tumor cells were grown in tissue culture flasks in Dubelco's medium with 10% fetal calf serum. Cells were harvested using Trypsin-EDTA solution (containing 0.05% trypsin), resuspended in PBS at 10,000,000/ml, and kept on ice.

1.1.2. Tumor Implantation

The mice were anesthetized with Nebutal (70 mg/kg). The back was shaved and prepared with alcohol solution. K1735-M2 melanoma cells were implanted by subcutaneous injection on the back with a 27-gage needle.

Approximately one million cells per mouse were injected. Mice were returned to their cages when fully awake and ambulatory.

1.1.3. After-Surgery Care

Each mouse was monitored daily. Signs of abnormal behavior or poor health were recorded. Abnormal conditions were reported to the study director for appropriate care. Tumor size was measured three times a week.

1.1.4. Mortality

Animals in the study were checked daily. Animals that appeared moribund or experiencing undue stress were humanely euthanized in a CO₂ chamber.

1.2. Treatment

1.2.1. Selection for Treatment

Animals with tumors were selected for treatment with the following criteria: tumors were growing and between 100 and 200 mm³.

Mice were weighed on the day of treatment and 1 week after treatment. Animals weighing greater or less than 20% the mean weight of all the animals on the day of treatment were removed from the study.

1.2.2. Dose

Animals were treated with a single i.v. injection (approximately 200 μL per mouse) as summarized in Table 1. Hist/Cit Buffer contains 50 mM histidine and 5 mM citrate at pH 7. Other samples include the anti-mouse VEGFR-2 antibody, a conjugate consisting of this antibody and the succinylated, dextran-coated polymerized vesicles described above (anti-VEGFR-2 antibody-dexPV) as well as an antibody conjugate containing yttrium-90 (anti-VEGFR-2 antibody-dexPV-Y90), a conjugate consisting of the dextran-coated polymerized vesicle and yttrium-90 (dexPV-Y90), and a conjugate consisting of the antibody, polymerized vesicle, and yttrium-90 (anti-VEGFR-2 antibody-PV-Y90),

TABLE 1
Doses for therapeutic agents targeted to VEGFR-2 and controls

| | | Antibody | PV | Y90 | |
|---|---|---|---|---|---|
| | | Dose | Dose | Dose | # of |
| Group | Sample | (μg/g) | (mg/g) | (μCi/g) | mice |
| 1 | Hist/Cit Buffer | NA | NA | NA | 9 |
| 2 | anti-VEGFR2 Antibody | 1 | NA | NA | 9 |
| 3 | anti-VEGFR2 Antibody- | 0.8 | 0.1 | NA | 9 |
| | dexPV | | | | |
| 4 | dexPV-Y90 | NA | 0.1 | 5 | 9 |
| 5 | anti-VEGFR2-Antibody- | 0.8 | 0.1 | 5 | 9 |
| | dexPV-Y90 | | | | |
| 6 | anti-VEGFr2-Antibody- | 2 | 0.1 | 5 | 9 |
| | PV-Y90 | | | | |

What is claimed is:

1. A lipid construct comprising a linking carrier, a targeting entity, and optionally a therapeutic entity.

2. The lipid construct of claim 1 wherein the linking carrier is selected from the group consisting of a polymerized liposome, liposome, polymer-coated liposome, and a micelle.

3. The lipid construct of claim 1, wherein the polymerizable lipid is 1,2-bis(10,12-tricosadiynoyl)-sn-glycero-3-phosphocholine.

4. The lipid construct of claim 1, wherein the polymerizable lipid is [PDA-PEG₃]₂-DTTA (compound 1, FIG. 3)

5. The lipid construct of claim 4, wherein said lipid chelator is 1,2-dimyristoyl-sn-glycero-3-phosphoethanolamidotriamine tetraacetic acid.

6. The lipid construct of claim 4, wherein the therapeutic entity is a metal ion.

7. The lipid construct of claim 4, wherein the metal ion is a radioactive metal ion.

8. The lipid construct of claim 4, wherein the metal ion is selected from the group consisting of Y-90, Bi-213, At-211, Cu-67, Sc-47, Ga-67, Rh-105, Pr-142, Nd-147, Pm-151, Sm-153, Ho-166, Gd-159, Tb-161, Eu-152, Er-171, Re-186, and Re-188.

9. The lipid construct of claim 4, wherein said therapeutic entity is ⁹⁰Y.

10. The lipid construct of claim 4, wherein said lipid chelator is N,N-bis[[[[(13′,15′-pentacosadiynamido-3,6-doxaoctyl)carbamoyl]methyl](carboxymethyl)amino]ethyl]glycine ([PDA-PEG₃]₂-DTTA₃).

11. The lipid construct of claim 4, wherein said lipid chelator contains a diacetylene lipid.

12. The lipid construct of claim 4, wherein said lipid chelator is selected from the group consisting of a derivative of diethylenetriaminepentaacetic acid, a derivative of ethylaminediaminetetracetic acid, and a derivative of 1,4,7,10-tetraazacyclododecane-N,N′,N″,N′″-tetraacetic acid (DOTA).

13. The lipid construct of claim 4, wherein said lipid chelator comprises an ionizable group selected from the group consisting of carboxyl, phosphate, phosphonate, sulfate, sulfonate, and sulfinate.

14. The lipid construct of claim 4, wherein said lipid chelator comprises a single ionizable group, said single ionizable group generating a surface capable of binding an isotope or metal with a valency of +2 or greater.

15. The lipid construct of claim 4, wherein said lipid chelator comprises a single ionizable group, said single ionizable group generating a surface capable of binding an isotope or metal with a valency of +3 or greater.

16. The lipid construct of claim 1, wherein said targeting entity is selected from the group consisting of a small molecule ligand and a protein.

17. The lipid construct of claim 1, wherein said targeting entity targets the lipid construct to a cell surface.

18. The lipid construct of claim 1, wherein the targeting entity is attached to the lipid construct through a group selected from the group consisting of amine, cyano, carboxylic acid, isothiocyanate, thiol, disulfide, α-halocarbonyl, α,β-unsaturated carbonyl and alkyl hydrazine.

19. The lipid construct of claim 1, wherein the targeting entity is attached to the lipid construct by non-covalent means.

20. The lipid construct of claim 19, wherein said non-covalent means is a biotin-avidin biotinylated antibody sandwich.

21. The lipid construct of claim 1, wherein said targeting entity is an antibody, protein, ligand, peptide, or nucleic acid.

22. The lipid construct of claim 1 wherein said targeting entity is VEGF or a derivative or portion thereof.

23. The lipid construct of claim 1, wherein said targeting entity is FGF or a derivative or portion thereof.

24. The lipid construct of claim 1, wherein said targeting entity is the peptide contains the sequence ATWLPPR, a derivative or homologue of ATWLPPR, or a peptidomimetic of a portion of this sequence.

25. The lipid construct of claim 1, wherein said targeting entity is an antibody against one or more of the VEGF receptors.

26. The lipid construct of claim 21, wherein said antibody is an anti-VEGFR-2 antibody or an anti-integrin alpha v subunit antibody.

27. The lipid construct of claim 26, wherein the therapeutic agent is selected from the group consisting of a radioisotope, prodrug, chemotherapeutic agent, toxin and a gene encoding a protein that exhibits cell toxicity.

28. The lipid construct of claim 21, wherein said antibody has a target selected from the group consisting of P-selectin, E-selectin, pleiotropin, chemokine and cytokine receptors, G-protein coupled receptors, endosialin, endoglin, VEGF receptor, PDGF receptor, FGF or EGF receptor, the matrix metalloproteases, and prostate specific membrane antigen (PSMA).

29. The lipid construct of claim 1, further comprising a stabilizing agent.

30. The lipid construct of claim 29, wherein the stabilizing agent is selected from the group consisting of dextran or aminodextran.

31. The lipid construct of claim 1, wherein the linking carrier is a polymerized liposome, the targeting entity is an anti-VEGFR-2 antibody or an anti-alpha v integrin subunit antibody, and the therapeutic entity is yttrium-90.

32. The lipid construct of claim 29, wherein the linking carrier is a polymerized liposome, the targeting entity is an anti-VEGFR-2 antibody or an anti-alpha v integrin subunit antibody, the therapeutic entity is ⁹⁰Y, and the stabilizing entity is aminodextran.
